(19) United States
(12) Patent Application Publication
(10) Pub. No.: US 2007/0253958 A1
(43) Pub. Date: Nov. 1, 2007

(54) Human monoclonal antibodies to FC gamma receptor II (CD32)

(75) Inventors: Jan G. J. Van De Winkel, Zeist (NL); Marcus Antonius Van Dijk, Bilthoven (NL); Arnout F. Gerritsen, Bunnik (NL)

Correspondence Address: LAHIVE & COCKFIELD, LLP, ONE POST OFFICE SQUARE, BOSTON, MA, 02109-2127, US

(73) Assignee: MEDAREX, INC., Princeton, NJ (US)

(21) Appl. No.: 11/731,771

(22) Filed: Mar. 30, 2007

Related U.S. Application Data
(63) Continuation of application No. PCT/US05/35055, filed on Sep. 29, 2005.
(60) Provisional application No. 60/615,429, filed on Sep. 30, 2004.

(51) Int. Cl. A61K 39/395; A01K 67/027; C07H 21/04; C07K 16/18; C12N 15/00; C12N 5/06

(52) U.S. Cl. 424/141.1; 435/320.1; 435/325; 435/326; 530/388.1; 536/23.1; 800/18

(57) ABSTRACT

The present invention provides isolated monoclonal antibodies, particularly human antibodies, that bind to CD32 with high affinity and have particular functional properties, such as inhibiting ligand binding, down-modulating surface expression of CD32 or specifically binding to the FcγRIIa-H131 allotype but not the FcγRIIa-R131 allotype. Nucleic acid molecules encoding the antibodies of the invention, expression vectors, host cells and methods for expressing the antibodies of the invention are also provided. Immunoconjugates, bispecific molecules, vaccine conjugates and pharmaceutical compositions comprising the antibodies of the invention are also provided. The invention also provides methods for detecting CD32, in particular for detecting the FcγRIIa-H131 allotype, methods of treatment using the antibodies of the invention, such as methods for treating autoimmune hemolytic anemia, and methods for enhancing antigen presentation using the antibodies of the invention.

### RELATED APPLICATIONS

This application is a continuation application of PCT/US2005/035055, filed Sep. 29, 2005, published pursuant to PCT Article 21 in English, which claims priority to U.S. Ser. No. 60/615,429 (filed Sep. 30, 2004), the entire contents of which are incorporated herein by this reference.

### BACKGROUND OF THE INVENTION

Receptors for the Fc region of antibodies (FcR) play a coordinating role in immunity. They are expressed on various types of cells and mediate functions ranging from endocytosis, phagocytosis, antibody-dependent cell-mediated cytotoxicity (ADCC), and cytokine production, to facilitation of antigen presentation. Antigen presentation represents a process in which antigens are captured, targeted to appropriate compartments, and processed before binding to major histocompatibility complex (MHC) molecules. FcγR molecules can potently enhance antigen presentation. The type of FcγR involved has been shown to be a crucial determinant for the types of epitopes presented by the antigen presenting cell (Amigorena, et al. (1998) J. Exp. Med. 187:505).

Leukocyte FcR for IgG (FcγR) comprises a multigene family, divided into three classes (FcγRI, II and III) based on differences in receptor structure, cell distribution, and affinity for IgG (Van de Winkel, et al. (1993) Immunol. Today 14:215). Eight genes have been identified, which encode various subclasses. FcγRII (CD32) represents a low-affinity receptor, interacting only with immune-complexed IgG. It is the only receptor with an ITAM signalling motif in its ligand-binding chain. FcγRIIa represents the most widely distributed FcγR subclass and is present on most myeloid cells, i.e., neutrophils, eosinophils, monocytes and macrophages, as well as on platelets (Deo, et al. (1997) Immunol. Today 18: 127; King, et al. (1990) Cell Immunol. 128:462; Daeron, M. (1997) Annu Rev Immunol. 15:203). FcγRIIb bears an ITIM inhibitory motif within its intracellular tail and is expressed on B-cells and phagocytes (Tridandapani, et al. (2002) J. Biol. Chem. 277:5082).

FcγRIIa is functionally polymorphic: a single nucleotide polymorphism results in either an arginine (R) or histidine (H) residue at position 131 in the membrane-proximal Ig-like domain. Amino acid 131 is located in the IgG-docking site and greatly affects receptor affinity for IgG immune complexes (Maxwell et al. (1999) Nat. Struct. Biol. 6:437-442; van der Pol et al. (2003) Immunogenetics 55:240-246). FcγRIIa-H131 exhibits a higher affinity for human IgG2 and IgG3 than FcγRIIa-R131. Notably, FcγRIIa-H131 represents the sole leukocyte FcγR capable of binding IgG2. The functionally different FcγRIIa alleles have been identified as risk factors for auto-immune and infectious diseases, as well as select polyneuropathies (van der Pol and van de Winkel (1998) Immunogenetics 48:222-232; van Sorge et al. (2003) Tissue Antigens 61:189-202). This polymorphism has also been linked to induction of side effects with therapeutic antibodies (Tax et al. (1997) Transplantation 63:106-112), and clinical efficacy of antibodies such as Rituxan® (Weng and Levy (2003) J. Clin. Oncol. 21:3940-3947).

The FcγRIIa-R131 and IIa-H131 allotypes have initially been determined functionally, based on the differential interaction of FcγRIIa allotypes with mouse IgG1 or human IgG2 antibodies in T-cell proliferation, EA-rosetting, or cellular cytotoxicity studies (van de Winkel et al. (1987) Scand. J. Immunol. 26:663-672; Clark et al. (1989) J. Immunol. 143:1731-1734; Parren et al. (1991) Res. Immunol. 142:749-763; Warmerdam et al. (1991) J. Immunol. 147:1338-1343; Wurflein et al. (1998) Cancer Res. 58:3051-3058). More recently, PCR-allotyping assays have been developed by a number of groups (Osborne et al. (1994) J. Immunol. Methods 173:207-217; Carlsson et al. (1998) Blood 92:1526-1531). However, both types of assays are not always applicable in routine laboratory settings.

In auto-immune hemolytic anemia (AIHA), antibodies against erythrocyte membrane antigens are present, leading to decreased survival of red blood cells (RBC). AIHA is frequently observed after allogeneous bone marrow transplantation due to allo-antibodies to ABO or minor RBC antigens (Drobyski, W. R. et al. (1996) Bone Marrow Transplant. 17:1093-1099; Hashimoto, C. (1998) Clin. Rev. Allergy Immunol. 16:285-295). In these cases, the response to conventional treatment is generally unsatisfactory, and prolonged courses of immunosuppressive therapy with corticosteroids, might influence engraftment and increase the risk for viral infections. Both FcγRI and FcγRII have been implicated in auto-immune cytopenic diseases in mice and man (Clynes, R. et al. (1995) Immunity 3:21-26; Kumpel, B. M. et al. (1990) Mol. Immunol. 27:247-256). FcγRIIa plays a role in clearance of immune-complexes, like human IgG-coated red blood cells in man (Dijstelbloem, H. M. et al. (2000) Arthritis Rheum. 43:2793-2800).

### SUMMARY OF THE INVENTION

The present invention provides isolated monoclonal antibodies, in particular human monoclonal antibodies, that bind to CD32 and that exhibit numerous desirable properties. These properties include high affinity binding to CD32 (FcγRII) but not to CD64 (FcγRI), CD16 (FcγRIII) or CD89 (FcαR), inhibition of ligand binding and down-modulation of surface expression of CD32. Furthermore, antibodies of the invention can inhibit autoimmune hemolytic anemia in animal models. Still further, certain antibodies of the invention have been shown to recognize the FcγRIIa-H131 allotype but not the FcγRIIa-R131 allotype and thus can be used to determine CD32 polymorphisms.

Accordingly, in one aspect, the invention pertains to an isolated human monoclonal antibody, or an antigen-binding portion thereof, wherein the antibody specifically binds to human FcγRII (CD32) and wherein the antibody exhibits at least one of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.

A preferred antibody of the invention exhibits all of the following properties:
a) binds FcγRIIa-H131, FcγRIIa-R131, FcγRIIb1*, but does not bind FcγRI (CD64), FcγRIII (CD16) or FcaR(CD89);
b) inhibits FcγRIIa ligand binding;
c) down-modulates surface expression of FcγRIIa; and
d) inhibits autoimmune hemolytic anemia.

Another preferred antibody of the invention exhibits the property of binding to FcγRIIa-H131 but not to FcγRIIa-R131 or FcγRIIb1*.

The antibodies of the invention can be, for example, full-length antibodies of an IgG1 isotype. Alternatively, the antibody can be an antibody fragment or a single chain antibody. The antibodies can be, for example, fully human, humanized or chimeric, but preferably are fully human antibodies.

In more preferred embodiments, the antibody binds to human CD32 with a K_{D} Of 5×10⁻⁸ M or less, binds to human CD32 with a K_{D} of 4×10⁻⁸ M or less, binds to human CD32 with a K_{D} of 3×10⁸ M or less, binds to human CD32 with a K_{D} of 2×10⁸ M or less, binds to human CD32 with a K_{D} of 1×10⁻⁸ M or less, or binds to human CD32 with a K_{D} of 9×10⁻⁹ M or less.

Another aspect of the invention pertains to an isolated monoclonal antibody, or an antigen-binding portion thereof, comprising a heavy chain variable region that is the product of or derived from a human V_{H} 3-33 or DP-44 gene, wherein the antibody specifically binds to human CD32. Yet another aspect pertains to an isolated monoclonal antibody, or an antigen-binding portion thereof, comprising a light chain variable region that is the product of or derived from a human V_{K} L18 or L15 gene, wherein the antibody specifically binds to human CD32. In a preferred embodiment, the isolated monoclonal antibody, or an antigen-binding portion thereof, comprising:
(a) a heavy chain variable region that is the product of or derived from a human V_{H} 3-33 or 3-13 gene; and
(b) a light chain variable region that is the product of or derived from a human Vk L18 or L15 gene;

wherein the antibody specifically binds to human CD32.

Particularly preferred combinations comprises a heavy chain variable region of a human V_{H} 3-33 gene and a light chain variable region of a human V_{K} L18 gene, or a heavy chain variable region of a human V_{H} DP-44 gene and a light chain variable region of a human V_{K} L15 gene.

Another aspect of the invention pertains to an isolated monoclonal antibody, or antigen binding portion thereof, comprising:
a heavy chain variable region that comprises CDR1, CDR2, and CDR3 sequences; and a light chain variable region that comprises CDR1, CDR2, and CDR3 sequences, wherein:
   (a) the heavy chain variable region CDR3 sequence comprises the amino acid sequence of SEQ ID NO: 3 or SEQ ID NO: 15, and conservative modifications thereof;
   (b) the light chain variable region CDR3 sequence comprises the amino acid sequence of SEQ ID NO: 6 or SEQ ID NO: 18, and conservative modifications thereof; and
   (c) the antibody specifically binds to human CD32.
      Preferably, the antibody exhibits at least one of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.
   More preferably, the heavy chain variable region CDR2 sequence comprises the amino acid sequence of SEQ ID NO: 2 or SEQ ID NO: 14, and conservative modifications thereof; and the light chain variable region CDR2 sequence comprises the amino acid sequence of SEQ ID NO: 5 or SEQ ID NO: 17, and conservative modifications thereof. Additionally or alternatively, the heavy chain variable region CDR1 sequence comprises the amino acid sequence of SEQ ID NO: 1 or SEQ ID NO: 13, and conservative modifications thereof; and the light chain variable region CDR1 sequence comprises the amino acid sequence of SEQ ID NO: 4 or SEQ ID NO: 16, and conservative modifications thereof.

Another aspect of the invention pertains to an isolated monoclonal antibody, or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NO: 7 and 19;
(b) the light chain variable region comprises an amino acid sequence that is at least 80% homologous to an amino acid sequence selected from the group consisting of SEQ ID NO: 8 and 20; and
(c) the antibody specifically binds to human CD32.
   Preferably, the antibody exhibits at least one of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.

In a preferred aspect, the invention provides an isolated monoclonal antibody, or antigen binding portion thereof, comprising:
(a) a heavy chain variable region CDR1 comprising the amino acid sequence of SEQ ID NO: 1 or SEQ ID NO: 13;
(b) a heavy chain variable region CDR2 comprising the amino acid sequence of SEQ ID NO: 2 or SEQ ID NO: 14;
(c) a heavy chain variable region CDR3 comprising the amino acid sequence of SEQ ID NO: 3 or SEQ ID NO: 15;
(d) a light chain variable region CDR1 comprising the amino acid sequence of SEQ ID NO: 4 or SEQ ID NO: 16;
(e) a light chain variable region CDR2 comprising the amino acid sequence of SEQ ID NO: 5 or SEQ ID NO: 17; and
(f) a light chain variable region CDR3 comprising the amino acid sequence of SEQ ID NO: 6 or SEQ ID NO: 18;
wherein the antibody specifically binds to human CD32.
   Preferably, the antibody exhibits at least one of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.
   A preferred antibody of the invention is an antibody comprising:
(a) a heavy chain variable region CDR1 comprising the amino acid sequence of SEQ ID NO: 1;
(b) a heavy chain variable region CDR2 comprising the amino acid sequence of SEQ ID NO: 2;
(c) a heavy chain variable region CDR3 comprising the amino acid sequence of SEQ ID NO: 3;
(d) a light chain variable region CDR1 comprising the amino acid sequence of SEQ ID NO: 4;
(e) a light chain variable region CDR2 comprising the amino acid sequence of SEQ ID NO: 5; and
(f) a light chain variable region CDR3 comprising the amino acid sequence of SEQ ID NO: 6.
   Another preferred antibody of the invention is an antibody comprising:
(a) a heavy chain variable region CDR1 comprising the amino acid sequence of SEQ ID NO: 13;
(b) a heavy chain variable region CDR2 comprising the amino acid sequence of SEQ ID NO: 14;
(c) a heavy chain variable region CDR3 comprising the amino acid sequence of SEQ ID NO: 15;
(d) a light chain variable region CDR1 comprising the amino acid sequence of SEQ ID NO: 16;
(e) a light chain variable region CDR2 comprising the amino acid sequence of SEQ ID NO: 17; and
(f) a light chain variable region CDR3 comprising the amino acid sequence of SEQ ID NO: 18.
   In yet another aspect, the invention provides an isolated monoclonal antibody, or antigen binding portion thereof comprising:
(a) a heavy chain variable region comprising an amino acid sequence selected from the group consisting of SEQ ID NO: 7 and 19; and
(b) a light chain variable region comprising an amino acid sequence selected from the group consisting of SEQ ID NO: 8 and 20;
wherein the antibody specifically binds to human CD32.
   A preferred antibody of the invention comprises:
(a) a heavy chain variable region comprising the amino acid sequence of SEQ ID NO:7; and
(b) a light chain variable region comprising the amino acid sequence of SEQ ID NO:8.
   Another preferred antibody of the invention comprises:
(a) a heavy chain variable region comprising the amino acid sequence of SEQ ID NO: 19; and
(b) a light chain variable region comprising the amino acid sequence of SEQ ID NO: 20.

Another aspect of the invention pertains to antibodies that compete for binding to CD32 with the specific antibodies of the invention disclosed herein.

The invention also provides immunoconjugates, comprising an antibody, or antigen-binding portion thereof, of the invention, linked to a second agent, such as a detectable marker or a cytotoxic agent. The invention also provides bispecific or multispecific molecules comprising an antibody, or antigen-binding portion thereof, of the invention, linked to a second functional moiety having a different binding specificity than the antibody, or antigen binding portion thereof, such as a second antibody or a cell receptor ligand.

bispecific or multispecific molecule of claim 36 and a pharmaceutically acceptable carrier. The invention also provides vaccine conjugates comprising an antibody, or antigen-binding portion thereof, of the invention linked to an antigen.

Pharmaceutical compositions, comprising an antibody, antibody fragment, immunoconjugate, bispecific or multispecific molecule or vaccine conjugate of the invention together with a pharmaceutically acceptable carrier are also encompassed by the invention.

In another aspect, the invention pertains to isolated nucleic acid molecules encoding the antibodies, or antigen-binding portions thereof, of the invention, as well as expression vectors comprising such nucleic acids and host cells comprising such expression vectors. A transgenic mouse comprising human immunoglobulin heavy and light chain transgenes, wherein the mouse expresses an antibody of the invention are also provided, as are hybridomas that produce such antibodies.

The invention also provides a method for detecting FcγRIIa-H131 in a sample, comprising:
a) contacting the sample with an antibody, or antigen-binding portion thereof, that binds FcγRIIa-H131 but does not bind FcγRIIa-R131; and
b) detecting the antibody, or antigen-binding portion thereof, bound to FcγRIIa-H131.
   In a preferred embodiment, the antibody is a human antibody, such as an antibody that comprises:
(a) a heavy chain variable region comprising the amino acid sequence of SEQ ID NO: 19; and
(b) a light chain variable region comprising the amino acid sequence of SEQ ID NO: 20.
   Binding of the antibody to FcγRIIa-H131 can be detected by, for example, flow cytometry or immunohistochemistry. In another embodiment, the detection method also includes contacting the sample with an antibody, or antigen-binding portion thereof, that binds FcγRIIa-R131 but does not bind FcγRIIa-H131.

In another aspect, the invention provides a method of treating or preventing autoimmune hemolytic anemia (AIHA) in a subject comprising administering to the subject the antibody, or antigen-binding portion thereof, of the invention such that the subject is treated for AIHA. The invention also provides a method of inhibiting growth of a target cell expressing CD32, comprising contacting the cell with an effective amount of an immunoconjugate of the invention such that growth of the target cell is inhibited. The invention also provides a method of inhibiting growth of a target cell expressing a target antigen, comprising contacting the cell with an effective amount of the bispecific or multispecific molecule of the invention such that growth of the target cell is inhibited. Still further, the invention provides a method of inducing or enhancing presentation of an antigen to an immune cell in a subject, comprising administering to the subject a vaccine conjugate of the invention such that presentation of the antigen is induced or enhanced.

The invention also pertains to a method for preparing an anti-CD32 antibody comprising:

(a) providing:
(i) a heavy chain variable region antibody sequence comprising a CDR1 sequence comprising the amino acid sequence of SEQ ID NO: 1 or 13, a CDR2 sequence comprising the amino acid sequence of SEQ ID NO: 2 or 14; and a CDR3 sequence comprising an amino acid sequence of SEQ ID NO: 3 or 15; or
(ii) a light chain variable region antibody sequence comprising a CDR1 sequence comprising the amino acid sequence of SEQ ID NO: 4 or 16, a CDR2 sequence comprising the amino acid sequence of SEQ ID NO: 5 or 17; and a CDR3 sequence comprising an amino acid sequence of SEQ ID NO: 6 or 18;

(b) altering at least one amino acid residue within at least one variable region antibody sequence, said sequence being selected from the heavy chain variable region antibody sequence and the light chain variable region antibody sequence, to create at least one altered antibody sequence; and

(c) expressing the altered antibody sequence as a protein.

Other features and advantages of the instant invention will be apparent from the following detailed description and examples which should not be construed as limiting. The contents of all references, Genbank entries, patents and published patent applications cited throughout this application are expressly incorporated herein by reference.

### BRIEF DESCRIPTION OF THE DRAWINGS

FIG. 1A shows the nucleotide sequence (SEQ ID NO: 9) and amino acid sequence (SEQ ID NO: 7) of the heavy chain variable region of the MDE-8 human monoclonal antibody. The CDR1 (SEQ ID NO: 1), CDR2 (SEQ ID NO: 2) and CDR3 (SEQ ID NO: 3) regions are delineated and the V, D and J germline derivations are indicated.

FIG. 1B shows the nucleotide sequence (SEQ ID NO: 10) and amino acid sequence (SEQ ID NO: 8) of the light chain variable region of the MDE-8 human monoclonal antibody. The CDR1 (SEQ ID NO: 4), CDR2 (SEQ ID NO: 5) and CDR3 (SEQ ID NO: 6) regions are delineated and the V and J germline derivations are indicated.

FIG. 2 shows the alignment of the amino acid sequence of the heavy chain variable region of MDE-8 (SEQ ID NO: 7) with the human-germline V_{H} 3-33 amino acid sequence (SEQ ID NO: 11).

FIG. 3 shows the alignment of the amino acid sequence of the light chain variable region of MDE-8 (SEQ ID NO: 8) with the human germline V_{K} L18 amino acid sequence (SEQ ID NO: 12).

FIG. 4A shows the nucleotide sequence (SEQ ID NO: 21) and amino acid sequence (SEQ ID NO: 19) of the heavy chain variable region of the MDE-9 human monoclonal antibody. The CDR1 (SEQ ID NO: 13), CDR2 (SEQ ID NO: 14) and CDR3 (SEQ ID NO: 15) regions are delineated and the V, D and J germline derivations are indicated.

FIG. 4B shows the nucleotide sequence (SEQ ID NO: 22) and amino acid sequence (SEQ ID NO: 20) of the light chain variable region of the MDE-9 human monoclonal antibody. The CDR1 (SEQ ID NO: 16), CDR2 (SEQ ID NO: 17) and CDR3 (SEQ ID NO: 18) regions are delineated and the V and J germline derivations are indicated.

FIG. 5 shows the alignment of the amino acid sequence of the heavy chain variable region of MDE-9 (SEQ ID NO: 19) with the human germline V_{H} DP-44 amino acid sequence (SEQ ID NO: 23).

FIG. 6 shows the alignment of the amino acid sequence of the light chain variable region of MDE-9 (SEQ ID NO: 20) with the human germline Vₖ L15 amino acid sequence (SEQ ID NO: 24).

FIG. 7A is a graph illustrating the binding of MDE-8 to a panel of transfected cells (expressing either FcγRIIa-R131, FcγRIIa-H131, FcγRIIb1*, CD64, CD16A, CD89 or CD20). The data was determined by flow cytometry and is expressed as mean fluorescence intensity. MDE-8 is represented by black bars. Open bars represent binding of a positive control mAb (mAb IV.3 for CD32 (FcγRIIa), mAb AT10 for CD32 (FcγRIIa and b), mAb H22 for CD64, mAb Gran-1 for CD16, mAb A77 for CD89, mAb B1 for CD20).

FIG. 7B is a graph illustrating the binding of MDE-8 to FcγRI- and FcγRIIa-expressing monocytic cell line THP-1, demonstrating that MDE-8 binds to FcγRIIa via the F(ab) portion. Binding of MDE-8 IgG (filled square), MDE-8 IgG in the presence of FcγRI-blocking mAb 197 (filled triangle), or MDE-8 F(ab′)2 fragments (open square) to THP-1 are shown. Data are representative of five experiments, yielding similar results.

FIG. 8 is a graph showing that MDE-8 inhibits the formation of EA-rosettes on human red blood cells. Human monocytes are shown as open bars and FcγRIIa transfected IIA1.6 cells are shown as gray bars. Negative controls were PBS and a human IgG1 CD89 antibody. Positive control was the CD32-blocking mAb IV.3. Data are representative of three individual experiments, yielding essentially identical results.

FIGS. 9A-9D are graphs showing that HuMAb MDE-8 downregulates cell surface FcγRIIa expression. THP-1 cells were incubated overnight with MDE-8 IgG (black squares), or MDE-8 F(ab′)₂ fragments (open squares) at different concentrations. As a control, cells were incubated with the humanized CD64 mAb H22 (black diamonds). Expression of FcγRIIa was assessed the next day with FITC-labeled mAb Fli8.26 (FIGS. 9A and 9C). FcγRI expression was assessed with FITC-labeled CD64 mAb 32.2 (FIGS. 9B and 9D). Levels of FcγRIIa and FcγRI on non-treated cells were set at 100%. Modulation is presented as percent decrease in receptor expression. Data represent means ±SD of 5 individual experiments, performed on separate days.

FIG. 10 is a graph showing that HuMAb MDE-8 inhibits the development of autoimmune hemolytic anemia in a mouse model. Eight week old female FcγRIIa transgenic mice or NTg mice (as a control) received an intravenous dose of MDE-8 (5 μg/g) or saline (control). After 60 min, mIgG1 anti-mouse erythrocyte antibody 105.2H was given intraperitoneally. Erythocyte counts were determined at various time-points using a Cell-Dyne 1700 multiparameter hematology analyser. Data represent means from four mice. ** P<0.01, * P<0.05.

FIGS. 11A-11D are graphs illustrating the binding characteristics of HuMab MDE-9. In FIG. 1A, IIA1.6 cells transfected with FcγRIIa-R131 or IIa-H131, were incubated with different concentrations of mAb MDE-9, CD32 mAb IV.3-FITC (positive control) or huIgG1 CD89 mAb (isotype control). Binding of MDE-9 was detected with FITC-labeled goat anti-human IgG-kappa. Data were analyzed by flow cytometry and represent five independent experiments, yielding similar results. In FIG. 11B, binding of MDE-9-FITC (10 μg/ml) was assesed on IIA1.6 transfectants. In FIG. 11C, binding of MDE-9-FITC (10 μg/ml) was assesed on isolated PMN and monocytes of healthy volunteers with either FcγRIIa-R/R131 or FcγRIIa-H/H 131 allotype. In FIG. 1 ID, IIA1.6 IIa-H131 transfectants were pre-incubated with FITC-labeled murine CD32 antibodies as indicated below the figure, and subsequently with MDE-9 (10 μg/ml). MDE-9 binding was detected by FITC-labeled goat anti-human IgG-kappa (n=5).

FIGS. 12A-12B are graphs illustrating the detection of FcγRIIa and FcγIIb on peripheral blood mononuclear cells using mAbs MDE-9 and 41H16. FIG. 12A shows expression of FcγRIIa and FcγIIb on peripheral blood mononuclear cells from H/H 131 donors (grey dots: PMN, black dots: monocytes and lymphocytes). In FIG. 12B, monocytes of FcγRIIa-H/H131 donors (n=6) were then incubated with pro-inflammatory (IFN-γ) and anti-inflammatory cytokines (IL-4 or IL-10) for 48 hours. The relative mean fluorescence intensities (MFI) of the inhibitory FcγRIIb (mAb 41H16) and the activating FcγRIIa-H131 (mAb MDE9) are expressed as percentages of expression levels on monocytes cultured with medium alone (set at 100%, dashed line). Mean values ±SD are given. Asterisks (*) mark significant changes compared to cultures in medium alone.

### DETAILED DESCRIPTION OF THE INVENTION

The present invention provides isolated monoclonal antibodies, in particular human monoclonal antibodies, that bind to CD32 and that exhibit numerous desirable properties. These properties include binding to CD32 (FcγRII) but not to CD64 (FcγRI), CD16 (FcγRIII) or CD89 (FcαR), inhibition of ligand binding and down-modulation of surface expression of CD32. Furthermore, antibodies of the invention can inhibit autoimmune hemolytic anemia in animal models. Still further, certain antibodies of the invention have been shown to recognize the FcγRIIa-H131 allotype but not the FcγRIIa-R131 allotype and thus can be used to determine CD32 polymorphisms.

In order that the present invention may be more readily understood, certain terms will be defined as follows. Additional definitions are set forth throughout the detailed description.

As used herein, the terms “CD32,” and “Fc-gamma receptor II” and “FcγRII” are used interchangeably and refer to a 40 kDa glycoprotein that is a low affinity receptor for IgG complexes and is expressed on a wide variety of cell types, including B lymphocytes, eosinophils, monocytes, granulocytes and platelets. The terms “FcγRIIa-H131” and “FcγRIIa-R131” refer to two allotypes having a histidine or an arginine, respectively, at amino acid position 131, and that differ in their ability to bind mouse IgG1 and human IgG2 complexes, as described further in Clark et al. (1989) J. Immunol. 143:1731-1734, Warmerdam et al. (1990) J. Exp. Med. 172:19-25 and Warmerdam et al. (1991) J. Immunol. 147:1338-1343.

As used herein, the term “FcγRIIb1” refers to a variant of the FcγRIIb1 isoform, which contains a single nucleotide difference as compared to FcγRIIb1 leading to an amino acid difference at position 11 of the cytoplasmic tail, as described further in Warmerdam et al. (1993) Int. Immunol. 3:239-247.

The terms “FcγR1” and “CD64” are used interchangeably to refer to the high-affinity receptor for IgG, which is constitutively expressed on antigen-presenting cells such as monocytes, macrophages, and dendritic cells.

The terms “FcγRIII” and “CD16” are used interchangeably and encompass the low affinity IgG receptors FcγRIIIa (CD16a) and FcγRIIIb (CD16b) that are expressed on NK cells and macrophages (for CD16a) and neutrophils (for CD16b).

The terms “FcαR” and “CD89” are used interchangeably and refer to the receptor for IgA.

The term “antibody” as referred to herein includes whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., CD32). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody which is substantially free of other antibodies having different antigenic specificities (e.g., an isolated antibody that specifically binds to CD32 is substantially free of antibodies that specifically bind antigens other than CD32). An isolated antibody that specifically binds to an epitope, isoform or variant of human CD32 may, however, have cross-reactivity to other related antigens, e.g., from other species (e.g., CD32 species homologs). Moreover, an isolated antibody may be substantially free of other cellular material and/or chemicals. In one embodiment of the invention, a combination of “isolated” monoclonal antibodies having different specificities are combined in a well defined composition.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “epitope” means a protein determinant capable of specific binding to, or specific binding by, an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “bispecific molecule” is intended to include any agent, e.g., a protein, peptide, or protein or peptide complex, which has two different binding specificities. For example, the molecule may bind to, or interact with, (a) a cell surface antigen and (b) an Fc receptor on the surface of an effector cell, e.g., CD32. The term “multispecific molecule” or “heterospecific molecule” is intended to include any agent, e.g., a protein, peptide, or protein or peptide complex, which has more than two different binding specificities. For example, the molecule may bind to, or interact with, (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to cell surface antigens, such as CD32, and to other targets, such as Fc receptors on effector cells.

The term “bispecific antibodies” also includes diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

As used herein, the term “heteroantibodies” refers to two or more antibodies, antibody binding fragments (e.g., Fab), derivatives therefrom, or antigen binding regions linked together, at least two of which have different specificities. These different specificities include a binding specificity for an Fc receptor on an effector cell, and a binding specificity for an antigen or epitope on a target cell, e.g., a tumor cell.

As used herein, a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

As used herein, “specific binding” refers to antibody binding to a predetermined antigen. Typically, the antibody binds with a dissociation constant (K_{D}) of 10⁻⁷ M or less, and binds to the predetermined antigen with a K_{D} that is at least two-fold less than its K_{D} for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen”.

As used herein, the term “high affinity” for an IgG antibody refers to an antibody having a K_{D} of 10⁻⁸ M or less, more preferably 10⁻⁹ M or less and even more preferably 10⁻¹⁰ M or less. However, “high affinity” binding can vary for other antibody isotypes. For example, “high affinity” binding for an IgM isotype refers to an antibody having a KD of 10⁻⁷ M or less, more preferably 10⁻⁸ M or less.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M).

As used herein, “isotype” refers to the antibody class (e.g., IgM or IgG1) that is encoded by heavy chain constant region genes.

As used herein, the term “effector cell” refers to an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Some effector cells express specific Fc receptors and carry out specific immune functions. In preferred embodiments, an effector cell is capable of inducing antibody-dependent cell-mediated cytotoxicity (ADCC), e.g., a neutrophil capable of inducing ADCC. For example, monocytes, macrophages, which express FcR are involved in specific killing of target cells and presenting antigens to other components of the immune system, or binding to cells that present antigens. In other embodiments, an effector cell can phagocytose a target antigen, target cell, or microorganism.

“Target cell” refers to any cell or pathogen whose elimination would be beneficial in a subject (e.g., a human or animal) and that can be targeted by a composition (e.g., a human monoclonal antibody, a bispecific, or a multi specific molecule) of the invention. For example, the target cell can be a cell expressing or overexpressing CD32. Alternatively, the target cell can be a tumor cell, such as a cell selected from cancer of the breast, ovarian, prostate, testicular, lung, colon, rectum, pancreas, liver, central nervous system, kidney, head, neck, bone, blood, and lymphatic system. In addition, target cells include auto-antibody producing lymphocytes (for treatment of autoimmune disease) and IgE-producing lymphocytes (for treatment of allergy). Target cells further include microorganisms (e.g., a bacterium or virus). Still other suitable targets include soluble antigens, such as rheumatoid factor and other auto-antibodies and toxins. Microorganisms include pathogens, viruses, bacteria, fungi, and protozoa.

The term “antigen” refers to any natural or synthetic immunogenic substance, such as a protein, peptide, or hapten. The term “antigen” also includes substances which are nonimmunogenic in uncomplexed form, but are immunogenic when complexed. The term “uncomplexed” includes substances which are not linked to form a molecular complex of the present invention. The term “complexed” includes substances which are linked to form a molecular complex of the present invention.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

As used herein, the terms “inhibits binding” and “blocks binding” (e.g., referring to inhibition/blocking of binding of CD32 ligand, e.g., IgG, to CD32) are used interchangeably and encompass both partial and complete inhibition/blocking. The inhibition/blocking of IgG to CD32 preferably reduces or alters the normal level or type of effector cell functions that occurs when IgG binds to CD32 without inhibition or blocking. Inhibition and blocking are also intended to include any measurable decrease in the binding affinity of IgG to CD32 when in contact with an anti-CD32 antibody as compared to the ligand not in contact with an anti-CD32 antibody, e.g., the blocking of CD32 ligands to CD32 by at least about 10%, 20%, 30%, 40%, 50%, 60%, 70%, 80%, 90%, 99%, or 100%.

The term “nucleic acid molecule”, as used herein, is intended to include DNA molecules and RNA molecules. A nucleic acid molecule may be single-stranded or double-stranded, but preferably is double-stranded DNA. The term “isolated nucleic acid molecule,” as used herein in reference to nucleic acids encoding antibodies or antibody portions (e.g., VH, VL, CDR3) that bind to CD32, is intended to refer to a nucleic acid molecule in which the nucleotide sequences encoding the antibody or antibody portion are free of other nucleotide sequences encoding antibodies or antibody portions that bind antigens other than CD32, which other sequences may naturally flank the nucleic acid in human genomic DNA.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, CHO cells, transfectomas, and lymphocytic cells.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The terms “transgenic, nonhuman animal” refers to a nonhuman animal having a genome comprising one or more human heavy and/or light chain transgenes or transchromosomes (either integrated or non-integrated into the animal's natural genomic DNA) and which is capable of expressing fully human antibodies. For example, a transgenic mouse can have a human light chain transgene and either a human heavy chain transgene or human heavy chain transchromosome, such that the mouse produces human anti-CD32 antibodies when immunized with CD32 antigen and/or cells expressing CD32. The human heavy chain transgene can be integrated into the chromosomal DNA of the mouse, as is the case for transgenic, e.g., HuMAb mice, or the human heavy chain transgene can be maintained extrachromosomally, as is the case for transchromosomal (e.g., KM) mice as described in WO 02/43478. Such transgenic and transchromosomal mice are capable of producing multiple isotypes of human monoclonal antibodies to CD32 (e.g., IgG, IgA and/or IgE) by undergoing V-D-J recombination and isotype switching.

Various aspects of the invention are described in further detail in the following subsections.

Anti-CD32 Antibodies

The antibodies of the invention are characterized by particular functional features or properties of the antibodies, examples of which include the features and properties described further in the Examples. For example, the antibodies bind specifically to human FcγRII (CD32). Preferably, an antibody of the invention binds to FcγRII (CD32) with high affinity, for example with a K_{D} of 10⁻⁸ M or less or 10⁻⁹ M or less or even 10⁻¹⁰ M or less. Thus, in one aspect, the invention provides an isolated human monoclonal antibody, or an antigen-binding portion thereof, wherein the antibody specifically binds to human FcγRII (CD32) and wherein the antibody exhibits at least one of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.
   An antibody of the invention can exhibit at least one of the above properties and may exhibit more than one property (although an antibody will not exhibit both properties a) and e) above).

In a preferred embodiment, the antibody exhibits all of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa; and
d) inhibits autoimmune hemolytic anemia.

In another preferred embodiment, the antibody exhibits the property of binding to FcγRIIa-H131 but not binding to FcγRIIa-R131 or FcγRIIb1*.

More preferably, the antibody binds to human CD32 with a K_{D} of 5×10⁻⁸ M or less, binds to human CD32 with a K_{D} of 4×10⁻⁸ M or less, binds to human CD32 with a K_{D} of 3×10⁻⁸ M or less, binds to human CD32 with a K_{D} of 2×10⁻⁸ M or less, binds to human CD32 with a K_{D} of 1×10⁻⁸ M or less, or binds to human CD32 with a K_{D} of 9×10⁻⁹ M or less.

The antibody can be a full-length antibody or an antibody fragment that retains its binding ability and the antibody can be of any isotype. In a preferred embodiment, the antibody is a full-length antibody of an IgG1 isotype. In another preferred embodiment, the antibody is an antibody fragment (e.g., a Fab fragment) or a single chain antibody (e.g., scFv).

Standard assays to evaluate the binding ability of the antibodies toward CD32 are known in the art, including for example, ELISA and flow cytometry. Suitable assays are described in detail in the Examples. The binding kinetics (e.g., binding affinity) of the antibodies also can be assessed by standard assays known in the art, such as by Biacore analysis. Other assays for evaluating the properties described above are described in detail in the Examples and include flow cytometric analyses to evaluate down-modulation of surface expression of FcγRIIa, EA-rosetting assays to evaluate inhibition of FcγRIIa ligand binding, and use of a human FcγRIIa transgenic mouse model of autoimmune hemolytic anemia (AIHA) to evaluate inhibition or prevention of AIHA by the antibody.

Monoclonal Antibodies MDE-8 and MDE-9

Preferred antibodies of the invention include the human monoclonal antibodies MDE-8 and MDE-9, isolated and structurally characterized as described in Examples 1 and 2. The V_{H} amino acid sequences of MDE-8 and MDE-9 are shown in SEQ ID NO: 7 and 19, respectively. The V_{L} amino acid sequences of MDE-8 and MDE-9 are shown in SEQ ID NO: 8 and 20, respectively.

Given that each of these antibodies can bind to CD32, the V_{H} and V_{L} sequences can be “mixed and matched” to create other anti-CD32 binding molecules of the invention. CD32 binding of such “mixed and matched” antibodies can be tested using the binding assays described above and in the Examples (e.g., ELISAs). Preferably, when V_{H} and V_{L} chains are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:

(a) a heavy chain variable region comprising an amino acid sequence selected from the group consisting of SEQ ID NO: 7 and 19; and

(b) a light chain variable region comprising an amino acid sequence selected from the group consisting of SEQ ID NO: 8 and 20;

wherein the antibody specifically binds human CD32.

Preferred heavy and light chain combinations include:

(a) a heavy chain variable region comprising the amino acid sequence of SEQ ID NO: 7; and (b) a light chain variable region comprising the amino acid sequence of SEQ ID NO: 8; or

(a) a heavy chain variable region comprising the amino acid sequence of SEQ ID NO: 19; and (b) a light chain variable region comprising the amino acid sequence of SEQ ID NO: 20.

In another aspect, the invention provides antibodies that comprise the heavy chain and light chain CDR1s, CDR2s and CDR3s of MDE-8 or MDE-9, or combinations thereof. The amino acid sequences of the MDE-8 V_{H} CDR1, 2 and 3 regions are shown in SEQ ID NOs: 1, 2 and 3, respectively. The amino acid sequences of the MDE-8 V_{L} CDR1, 2 and 3 regions are shown in SEQ ID NOs: 4, 5 and 6, respectively. The amino acid sequences of the MDE-9 V_{H} CDR1, 2 and 3 regions are shown in SEQ ID NOs: 13, 14 and 15, respectively. The amino acid sequences of the MDE-9 V_{L} CDR1, 2 and 3 regions are shown in SEQ ID NOs: 16, 17 and 18, respectively. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Accordingly, in another aspect, the invention provides an isolated monoclonal antibody, or antigen binding portion thereof comprising:
(a) a heavy chain variable region CDR1 comprising the amino acid sequence of SEQ ID NO: 1 or SEQ ID NO: 13;
(b) a heavy chain variable region CDR2 comprising the amino acid sequence of SEQ ID NO: 2 or SEQ ID NO: 14;
(c) a heavy chain variable region CDR3 comprising the amino acid sequence of SEQ ID NO: 3 or SEQ ID NO: 15;
(d) a light chain variable region CDR1 comprising the amino acid sequence of SEQ ID NO: 4 or SEQ ID NO: 16;
(e) a light chain variable region CDR2 comprising the amino acid sequence of SEQ ID NO: 5 or SEQ ID NO: 17; and
(f) a light chain variable region CDR3 comprising the amino acid sequence of SEQ ID NO: 6 or SEQ ID NO: 18;
wherein the antibody specifically binds to human CD32.
   In a preferred embodiment, the antibody exhibits at least one of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.
   A preferred antibody of the invention comprises:
(a) a heavy chain variable region CDR1 comprising the amino acid sequence of SEQ ID NO: 1;
(b) a heavy chain variable region CDR2 comprising the amino acid sequence of SEQ ID NO: 2;
(c) a heavy chain variable region CDR3 comprising the amino acid sequence of SEQ ID NO: 3;
(d) a light chain variable region CDR1 comprising the amino acid sequence of SEQ ID NO: 4;
(e) a light chain variable region CDR2 comprising the amino acid sequence of SEQ ID NO: 5; and
(f) a light chain variable region CDR3 comprising the amino acid sequence of SEQ ID NO: 6.
   Another preferred antibody of the invention comprises:
(a) a heavy chain variable region CDR1 comprising the amino acid sequence of SEQ ID NO: 13;
(b) a heavy chain variable region CDR2 comprising the amino acid sequence of SEQ ID NO: 14;
(c) a heavy chain variable region CDR3 comprising the amino acid sequence of SEQ ID NO: 15;
(d) a light chain variable region CDR1 comprising the amino acid sequence of SEQ ID NO: 16;
(e) a light chain variable region CDR2 comprising the amino acid sequence of SEQ ID NO: 17; and
(f) a light chain variable region CDR3 comprising the amino acid sequence of SEQ ID NO: 18.
   Antibodies Having Particular Germline Sequences

In certain embodiments, an antibody of the invention comprises a heavy chain variable region from a particular germline heavy chain immunoglobulin gene and/or a light chain variable region from a particular germline light chain immunoglobulin gene.

For example, in a preferred embodiment, the invention provides an isolated monoclonal antibody, or an antigen-binding portion thereof, comprising a heavy chain variable region that is the product of or derived from a human V_{H} 3-33 gene, wherein the antibody specifically binds to human CD32. In another preferred embodiment, the invention provides an isolated monoclonal antibody, or an antigen-binding portion thereof, comprising a heavy chain variable region that is the product of or derived from a human V_{H} DP-44 gene, wherein the antibody specifically binds CD32. In another preferred embodiment, the invention provides an isolated monoclonal antibody, or an antigen-binding portion thereof, comprising a light chain variable region that is the product of or derived from a human V_{H} L18 gene, wherein the antibody specifically binds to human CD32. In another preferred embodiment, the invention provides an isolated monoclonal antibody, or an antigen-binding portion thereof, comprising a light chain variable region that is the product of or derived from a human V_{H} L15 gene, wherein the antibody specifically binds to human CD32.

In yet another preferred embodiment, the invention provides an isolated monoclonal antibody, or an antigen-binding portion thereof, wherein the antibody:

(a) comprises a heavy chain variable region that is the product of or derived from a human V_{H} 3-33 or DP-44 gene (which encodes the amino acid sequence set forth in SEQ ID NOs: 11 and 23, respectively);

(b) comprises a light chain variable region that is the product of or derived from a human Vₖ L18 or L15 gene (which encode the amino acid sequences set forth in SEQ ID NOs: 12 and 24, respectively); and

(c) specifically binds to human CD32.

An example of an antibody having V_{H} and V_{K} of V_{H} 3-33 and Vk L18, respectively, is the MDE-8 antibody. An example of an antibody having V_{H} and V_{K} of VH DP-44 and Vk L15, respectively, is the MDE-9 antibody.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins (eg., using the Vbase database) and selecting the human germline immunoglobulin sequence that is closest in sequence (i.e., greatest % identity) to the sequence of the human antibody. A human antibody that is “the product of” or “derived from” a particufar human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Homologous Antibodies

In yet another embodiment, an antibody of the invention comprises heavy and light chain variable regions comprising amino acid sequences that are homologous to the amino acid sequences of the preferred antibodies described herein, and wherein the antibodies retain the desired functional properties of the anti-CD32 antibodies of the invention.

For example, the invention provides an isolated monoclonal antibody, or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NO: 7 and 19;
(b) the light chain variable region comprises an amino acid sequence that is at least 80% homologous to an amino acid sequence selected from the group consisting of SEQ ID NO: 8 and 20; and
(c) the antibody specifically binds to human CD32.
   In one embodiment, such an antibody can exhibit one or more of the following properties:
a) binds FcγRIIa-H131, FcγRIIa-R131, FcγRIIb1*, but does not bind FcγRI (CD64), FcγRIII (CD 16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.

In various embodiments, the antibody can be, for example, a human antibody, a humanized antibody or a chimeric antibody. Preferably, the antibody binds to human CD32 with a K_{D} of 9×10⁻⁹ M or less.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs: 7, 8, 9, or 10, followed by testing of the encoded altered antibody for retained function (i.e., the functions set forth in (c) and (d) above) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., MDE-8 or MDE-9), or conservative modifications thereof, and wherein the antibodies retain the desired functional properties of the anti-CD32 antibodies of the invention. Accordingly, the invention provides an isolated monoclonal antibody, or antigen binding portion thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises the amino acid sequence of SEQ ID NO: 3 or 15, and conservative modifications thereof;
(b) the light chain variable region CDR3 sequence comprises the amino acid sequence of SEQ ID NO: 6 or 18, and conservative modifications thereof; and
(c) the antibody specifically binds to human CD32.
   In one embodiment, such an antibody can exhibit one or more of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.

Preferably, the antibody binds to human CD32 with a K_{D} of 9×10⁻⁹ M or less. In a preferred embodiment, the heavy chain variable region CDR2 sequence comprises the amino acid sequence of SEQ ID NO: 2 or 14, and conservative modifications thereof; and the light chain variable region CDR2 sequence comprises the amino acid sequence of SEQ ID NO: 5 or 17, and conservative modifications thereof. In another preferred embodiment, the heavy chain variable region CDR1 sequence comprises the amino acid sequence of SEQ ID NO: 1 or 13, and conservative modifications thereof; and the light chain variable region CDR1 sequence comprises the amino acid sequence of SEQ ID NO: 4 or 16, and conservative modifications thereof.

In various embodiments, the antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (i) through (iv) above) using the functional assays described herein.

Antibodies that Bind to the Same Epitope as Anti-CD32 Antibodies of the Invention

In another embodiment, the invention provides antibodies that bind to the same epitope as do the various anti-CD32 antibodies of the invention provided herein, such as other human antibodies that bind to the same epitope as the MDE-8 antibody (having VH and VL sequences as shown in SEQ ID NOs: 7 and 8, respectively) or the MDE-9 antibody (having VH and VL sequences as shown in SEQ ID NOs: 19 and 20, respectively), described herein. Such additional antibodies can be identified based on their ability to cross-compete (e.g., to competitively inhibit the binding of, in a statistically significant manner) with other antibodies of the invention, such as MDE-8 or MDE-9, in standard CD32 binding assays. The ability of a test antibody to inhibit the binding of, e.g., MDE-8 or MDE-9 to human CD32 demonstrates that the test antibody can compete with that antibody for binding to human CD32; such an antibody may, according to non-limiting theory, bind to the same or a related (e.g., a structurally similar or spatially proximal) epitope on human CD32 as the antibody with which it competes. In a preferred embodiment, the antibody that binds to the same epitope on human CD32 as MDE-8 or MDE-9 is a human monoclonal antibody. Such human monoclonal antibodies can be prepared and isolated as described in the Examples.

Engineered and Modified Antibodies

An antibody of the invention further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.).

Accordingly, another embodiment of the invention pertains to an isolated monoclonal antibody, or antigen binding portion thereof, comprising a heavy chain variable region comprising CDR1, CDR2, and CDR3 sequences of SEQ ID NOs: 1, 2, and 3, respectively, or of SEQ ID NOs: 13, 14 and 15, respectively, and a light chain variable region comprising CDR1, CDR2, and CDR3 sequences comprising an amino acid sequence of SEQ ID NOs: 4, 5, and 6, respectively, or of SEQ ID NOs: 16, 17 and 18, respectively. Thus, such antibodies contain the V_{H} and V_{L} CDR sequences of monoclonal antibody MDE-8 or MDE-9 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference.

Preferred framework sequences for use in the antibodies of the invention are those that are structurally similar to the framework sequences used by selected antibodies of the invention, e.g., similar to the V_{H} 3-33 or DP-44 sequences (SEQ ID NO: 11 or 23) and/or the Vₖ L18 or L15 framework sequence (SEQ ID NO: 12 or 24) used by preferred monoclonal antibodies of the invention. The V_{H} CDR1, 2 and 3 sequences, and the V_{K} CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

Accordingly, in another embodiment, the invention provides isolated anti-CD32 monoclonal antibodies, or antigen binding portions thereof, comprising a heavy chain variable region comprising: (a) V_{H} CDR1, CDR2, and CDR3 regions comprising the amino acid sequences of SEQ ID NOs: 1, 2, and 3, respectively, or SEQ ID NOs: 13, 14 and 15, respectively, or an amino acid sequence having one, two, three, four or five amino acid substitutions, deletions or additions as compared to SEQ ID NOs: 1, 2, and 3, or SEQ ID NOs: 13, 14 and 15; (b) VK CDR1, CDR2, and CDR3 regions comprising the amino acid sequences of SEQ ID NOs: 4, 5, and 6, respectively, or SEQ ID NOs: 16, 17 and 18, respectively, or an amino acid sequence having one, two, three, four or five amino acid substitutions, deletions or additions as compared to SEQ ID NOs: 4, 5, and 6, or SEQ ID NOs: 16, 17 and 18.

Engineered antibodies of the invention include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. For example, for MDE-8, amino acid residue #3 (within FR1) of V_{H} is a histidine whereas this residue in the corresponding V_{H} 3-33 germline sequence is a glutamine. As another example, for MDE-9, amino acid residue #28 (within FR1) of V_{H} is an alanine whereas this residue in the corresponding V_{H} DP-44 germline sequence is a theonine. To return the framework region sequences to their germline configuration, the somatic mutations can be “backmutated” to the germline sequence by, for example, site-directed mutagenesis or PCR-mediated mutagenesis (e.g., residue 3 of FR1 of the V_{H} of MDE-8 can be “backmutated” from histidine to glutamine or residue 28 of FR1 of the V_{H} of MDE-9 can be “backmutated” from alanine to threonine). Such “backmutated” antibodies are also intended to be encompassed by the invention.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

Methods of Engineering Antibodies

As discussed above, the anti-CD32 antibodies having V_{H} and V_{K} sequences disclosed herein can be used to create new anti-CD32 antibodies by modifying the VH and/or V_{K} sequences, or the constant region(s) attached thereto. Thus, in another aspect of the invention, the structural features of an anti-CD32 antibody of the invention, e.g. MDE-8 or MDE-9, are used to create structurally related anti-CD32 antibodies that retain at least one functional property of the antibodies of the invention, such as binding to human CD32. For example, one or more CDR regions of MDE-8, MDE-9, or mutations thereof, can be combined recombinantly with known framework regions, and/or other CDRs to create additional, recombinantly-engineered, anti-CD32 antibodies of the invention, as discussed above. Other types of modifications include those described in the previous section. The starting material for the engineering method is one or more of the V_{H} and/or V_{K} sequences provided herein, or one or more CDR regions thereof. To create the engineered antibody, it is not necessary to actually prepare (i.e., express as a protein) an antibody having one or more of the V_{H} and/or V_{K} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing an anti-CD32 antibody comprising:

(a) providing:
(i) a heavy chain variable region antibody sequence comprising a CDR1 sequence comprising the amino acid sequence of SEQ ID NO: 1 or 13, a CDR2 sequence comprising the amino acid sequence of SEQ ID NO: 2 or 14; and a CDR3 sequence comprising an amino acid sequence of SEQ ID NO: 3 or 15; and/or
(ii) a light chain variable region antibody sequence comprising a CDR1 sequence comprising the amino acid sequence of SEQ ID NO: 4 or 16, a CDR2 sequence comprising the amino acid sequence of SEQ ID NO: 5 or 17; and a CDR3 sequence comprising an amino acid sequence of SEQ ID NO: 6 or 18;

(b) altering at least one amino acid residue within at least one variable region antibody sequence, said sequence being selected from the heavy chain variable region antibody sequence and the light chain variable region antibody sequence, to create at least one altered antibody sequence; and

(c) expressing the altered antibody sequence as a protein.

Standard molecular biology techniques can be used to prepare and express the altered antibody sequence.

Preferably, the antibody encoded by the altered antibody sequence(s) is one that retains one, some or all of the functional properties of the anti-CD32 antibodies described herein, which functional properties include, but are not limited to:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R1131 or FcγRIIb1*.

The functional properties of the altered antibodies can be assessed using standard assays available in the art and/or described herein, such as those set forth in the Examples (e.g., flow cytometry, binding assays, EA-rosetting and the like).

In certain embodiments of the methods of engineering antibodies of the invention, mutations can be introduced randomly or selectively along all or part of an anti-CD32 antibody coding sequence and the resulting modified anti-CD32 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

Nucleic Acid Molecules Encoding Antibodies of the Invention

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Preferred nucleic acids molecules of the invention are those encoding the VH and VL sequences of the MDE-8 and MDE-9 monoclonal antibodies. The DNA sequence encoding the VH sequence of MDE-8 is shown in SEQ ID NO: 9. The DNA sequence encoding the VL sequence of MDE-8 is shown in SEQ ID NO: 10. The DNA sequence encoding the VH sequence of MDE-9 is shown in SEQ ID NO: 21. The DNA sequence encoding the VL sequence of MDE-9 is shown in SEQ ID NO: 22.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH₁, CH₂ and CH₃). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Production of Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against CD32 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Accordingly, the mice exhibit reduced expression of mouse IgM or K, and in response to immunization, the introduced human heavy and light chain transgenes undergo class switching and somatic mutation to generate high affinity human IgGK monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise anti-CD32 antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-CD32 antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-CD32 antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Immunization of Human Ig Mice

When human Ig mice are used to raise human antibodies of the invention, such mice can be immunized with a purified or enriched preparation of CD32 antigen and/or recombinant CD32, or an CD32 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of CD32 antigen can be used to immunize the human Ig mice intraperitoneally.

Detailed procedures to generate fully human monoclonal antibodies to CD32 are described in Example 1 below. Cumulative experience with various antigens has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-CD32 human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo 12).

Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

Generation of Transfectomas Producing Monoclonal Antibodies of the Invention

Antibodies of the invention also can be produced in a host cell transfectoma using, for example, a combination of well known recombinant DNA techniques and gene transfection methods (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRc promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Pro karyotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In addition, or alternatively, to simply binding CD32, engineered antibodies such as those described above may be selected for their retention of other functional properties of antibodies of the invention, such as:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.
   Characterization of Antibody Binding to Antigen

Antibodies of the invention can be tested for binding to CD32 by, for example, standard ELISA. Briefly, microtiter plates are coated with purified CD32 at 0.25 μg/ml in PBS, and then blocked with 5% bovine serum albumin in PBS. Dilutions of antibody (e.g., dilutions of plasma from CD32-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with CD32 immunogen. Hybridomas that bind with high avidity to CD32 are subcloned and further characterized. One clone from each hybridoma, which retains the reactivity of the parent cells (by ELISA), can be chosen for making a 5-10 vial cell bank stored at −140° C., and for antibody purification.

To purify anti-CD32 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected anti-CD32 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using CD32 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-CD32 human IgGs can be further tested for reactivity with CD32 antigen by Western blotting. Briefly, CD32 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Immunoconjugates

In another aspect, the present invention features an anti-CD32 antibody, or a fragment thereof, conjugated to a diagnostic or therapeutic moiety, such as a detectable marker, a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Non-limiting examples of detectable markers to which an antibody can be conjugated include fluorescein, cyanin, Cy-3, biotin and the like. Antibodies can be labeled with such detectable markers by methods known in the art, including the techniques described in the Examples.

Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg™; Wyeth-Ayerst).

Cytoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Method for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the antibodies of the invention.

The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Bispecific Molecules

In another aspect, the present invention features bispecific molecules comprising an anti-CD32 antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, tumor specific or pathogen specific antigens, peptide or binding mimetic, such that a bispecific molecule results.

Accordingly, the present invention includes bispecific molecules comprising at least one first binding molecule having specificity for CD32 and a second binding molecule having specificity for a second target epitope. In a particular embodiment of the invention, the second binding molecule may be another antibody or antibody portion specific for a target antigen on a target cell, for example, a tumor cell or a pathogen. As an example, the second binding molecule may be an anti-Her2/Neu antibody, which binds breast cancer cells. In another particular embodiment of the invention, the second binding molecule may be a ligand specific for a target receptor. As an example, the second binding molecule may be EGF or the receptor binding portion of epidermal growth factor (EGF), which binds EGF receptor on tumor cells. Therefore, the invention includes bispecific molecules capable of binding both to FcγRIIa expressing effector cells and to target cells. These bispecific molecules target CD32 expressing effector cells to target cells expressing a target molecule to which the bispecific molecule binds and triggers Fc receptor-mediated effector cell activities, such as phagocytosis of target-expressing cells, antibody dependent cell-mediated cytotoxicity (ADCC), cytokine release, or generation of superoxide anion.

In various embodiments, the second moiety of the bispecific molecule can specificity for a target epitope of an antigen selected from the following: anthrax antigens, botulism toxin, malaria antigens, equine encephalitis virus antigen, Y. peslis antigens, gastrin releasing peptide receptor antigen (GRP), mucin antigens, epidermal growth factor receptor (EGF-R), HER2/neu, HER3, HER4, CD20, CD30, PSMA, carcinoembryonic antigen (CEA), Pmel17, beta-human chorionic gonadotropin (βHCG), alpha-fetoprotein (AFP), gp100, MART1, TRP-2, melan-A, NY-ESO-1, MN (gp250) idiotype, MAGE antigens, SART antigens, Tyrosinase, Telomerase, TAG-72 antigen, MUC-1 antigens, the blood group antigens Lea, Leb, LeX, LeY, H-2, B-1, and B-2, HIV-1 gag, HIV-1 env, HIV-1 nef, HBV core, FAS, HSV-1, HSV-2, p17, HTLV, FELV, ORF2 and ORF3 antigens, protozoan-specific antigens, Candida albicans antigen, bacterial antigens, Toxoplasma gondii antigen, Treponema pallidum antigen, Staphylococcus aureus antigen, Streptococcus hemolyticus antigen, and Mycobacterium tuberculsis antigen.

In an embodiment of the invention in which the bispecific molecule is multispecific, the molecule can further include a third binding specificity, in addition to an anti-Fc binding specificity and an anti-CD32 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the F_{C} receptor or target cell antigen. The “anti-enhancement factor portion” can bind an F_{C} receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one embodiment, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In one embodiment, the binding specificity for an Fcγ receptor (eg. anti-CD32 antibody of the invention) is provided by a monoclonal antibody, the binding of which is not blocked by human immunoglobulin G (IgG).

While human monoclonal antibodies are preferred, other antibodies which can be employed in the bispecific molecules of the invention are murine, chimeric and humanized monoclonal antibodies.

The bispecific molecules of the present invention can be prepared by conjugating the constituent binding specificities, e.g., the anti-CD32 binding specificity and anti-target cell binding specificity, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γcounter or a scintillation counter or by autoradiography.

Antibody Vaccine Conjugates

The present invention further provides a variety of therapeutic conjugates which include one or more human anti-CD32 antibodies (or fragments thereof) linked to one or more antigens, such as a tumor or viral antigen, to form a vaccine conjugate. This allows for targeting of a wide variety of antigens to CD32-expressing immune cells, particularly antigen presenting cells (APCs), to enhance processing, presentation and, ultimately, an immune response against the antigen(s).

Antibody-antigen vaccine conjugates of the invention can be made using any practical methodology, including genetically or chemically. In any case, the antibody portion of the conjugate may an consist of the whole antibody or a portion of the antibody, such as the Fab fragment or single-chain Fv. In addition, more than one antigen can be added to a single antibody construct.

Genetically constructed anti-CD32 antibody-antigen conjugates (e.g., those expressed as a single recombinant fusion protein) can be made by linking the antigen of choice to the antibody at a variety of locations. For example, the antigen can be fused to the end of the CH₃ domain of the human antibody heavy chain. The antigen also can be fused at the hinged region of the antibody heavy chain in Fab-fusion constructs, or in sequence with the variable light and heavy chains (V_{H} and V_{L}) in single chain fusion constructs (ScFv constructs). Alternatively, the antigen can be fused to the antibody light chain instead of the antibody heavy chain.

Chemically constructed antibody-antigen conjugates can be made using a variety of well known and readily available cross-linking reagents. These cross-linking reagents can be homofunctional or heterofunctional compounds, such as SPDP, SATA, SMCC, DTNB, that form covalent linkages with different reactive amino acid or carbohydrate side chains on the anti-CD32 antibody and selected antigen.

Any antigen that can be cloned and expressed or purified can be selected for use in the antibody-antigen vaccine conjugates of the present invention. Techniques for obtaining such antigens are well-known in the art. For example, tumor-associated antigens can be directly purified from cancer cells and identified by physiochemical techniques such as tandem mass spectrometry. Alternatively, tumor-specific T-cell clones can be tested against antigen-negative cells that have acquired antigen by being transfected with plasmid DNA clones to isolate the clone expressing the antigen. Synthetic peptides can then be constructed to precisely identify the antigenic site or epitope.

A significant advantage of the antibody-antigen conjugates of the present invention is their ability to rapidly elicit strong immune responses from vaccines to thereby improve the efficacy of vaccination. Accordingly, infectious disease antigens and tumor antigens against which immune responses are protective or therapeutic can be conjugated to human anti-CD32 antibodies of the invention, such as antibody MDE-8 or MDE-9, to form highly effective vaccines. Examples of infectious disease antigens include, but are not limited to, viral proteins, bacterial proteins and carbohydrates, fungal proteins and carbohydrates.

Antibody-antigen conjugates of the invention also can be used to improve the efficacy of vaccination against infectious organisms and their toxins that may be encountered during travel or through biowarfare. Examples of such antigens include, for example, anthrax antigens, botulism toxin, malaria antigens, equine encephalitis, and Y. pestis antigens.

Other suitable antigens for use in the antibody-antigen conjugates of the invention include tumor-associated antigens for the prevention or treatment of cancers. Examples of tumor-associated antigens include, but are not limited to, gastrin releasing peptide receptor antigen (GRP), mucin antigens, epidermal growth factor receptor (EGF-R), HER2/neu, HER3, HER4, CD20, CD30, PSMA, carcinoembryonic antigen (CEA), Pmel17, beta-human chorionic gonadotropin (PHCG), alpha-fetoprotein (AFP), gp 100, MART1, TRP-2, melan-A, NY-ESO-1, MN (gp250) idiotype, MAGE antigens, e.g. MAGE-1 and MAGE-3, SART antigens, Tyrosinase, Telomerase, TAG-72 antigen, and MUC-1 antigens. Tumor associated antigens also include the blood group antigens, for example, Le^{a}, Le^{b}, LeX, LeY, H-2, B-1, B-2 antigens. In another preferred embodiment, more than one antigen is fused to a single anti-CD32 antibody construct. For example, a MAGE antigen can be combined with other antigens such as melanin A, tyrosinase, and gp100 along with adjuvants such as GM-CSF or IL-12, and fused to an anti-CD32 antibody construct, e.g., MDE-8 or MDE-9.

Other suitable antigens include viral antigens for the prevention or treatment of viral diseases. Examples of viral antigens include, but are not limited to, HIV-1 gag, HIV-1 env, HIV-1 nef, HBV core, FAS, HSV-1, HSV-2, p17, HTLV, FELV, ORF2 and ORF3 antigens. In another preferred embodiment, the selected antigen is a melanoma-specific antigen including, but not limited to, gp100 or Pmel17. In another preferred embodiment, the selected antigen is a protozoan-specific antigen, for example, a fungal antigen (e.g., Candida albicans). In yet another embodiment, the selected antigen is a bacterial antigen including, but not limited to, Toxoplasma gondii or Treponema pallidum. The antibody-bacterial antigen conjugates of the invention can be in the treatment or prevention of various bacterial diseases such as Anthrax, Botulism, Tetanus, Chlamydia, Cholera, Diphtheria, Lyme Disease, Syphilis and Tuberculosis (e.g., Staphylococcus aureus, Streptococcus hemolyticus, and Mycobacterium tuberculsis).

Pharmaceutical Compositions

In another aspect, the present invention provides a composition, e.g., a pharmaceutical composition, containing one or a combination of monoclonal antibodies, or antigen-binding portion(s) thereof, of the present invention, formulated together with a pharmaceutically acceptable carrier. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecifics) that bind to different epitopes on the target antigen or that have complementary activities.

Pharmaceutical compositions of the invention also can be administered in combination therapy, i.e., combined with other agents. For example, the combination therapy can include an anti-CD32 antibody of the present invention combined with at least one other anti-inflammatory or immunosuppressant agent. Examples of therapeutic agents that can be used in combination therapy are described in greater detail below in the section on uses of the antibodies of the invention.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjuage, or bispecific molecule, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-CD32 antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-CD32 antibody of the invention preferably results in a decrease in severity of disease symptoms, an increase in frequency and duration of disease symptom-free periods, or a prevention of impairment or disability due to the disease affliction. For example, for the treatment of AIHA, a “therapeutically effective dosage” preferably inhibits disease symptoms by about 20%, more preferably by at least about 40%, even more preferably by at least about 60%, and still more preferably by at least about 80% relative to untreated subjects. The ability of a compound to inhibit AIHA can be evaluated in an animal model system, such as that described in the Examples. Alternatively, this property of a composition can be evaluated by examining the ability of the compound to inhibit, such inhibition in vitro by assays known to the skilled practitioner. One of ordinary skill in the art would be able to determine such amounts based on such factors as the subject's size, the severity of the subject's symptoms, and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBSLeft. 346:123; J. J. Killion; I. J. Fidler (1994) Immunomethods 4:273.

Uses and Methods of the Invention

The human antibodies, antibody compositions and methods of the present invention have numerous in vitro and in vivo diagnostic and therapeutic utilities involving the detection, diagnosis and/or treatment of disorders involving CD32. For example, these molecules can be administered to cells in culture, e.g. in vitro or ex vivo, or to human subjects, e.g., in vivo, to treat, prevent and to diagnose a variety of disorders. As used herein, the term “subject” is intended to include human and non-human animals. Non-human animals includes all vertebrates, e.g., mammals and non-mammals, such as non-human primates, sheep, dogs, cats, cows, horses, pigs, chickens, avians, amphibians, and reptiles. When antibodies to CD32 are administered together with another agent, the two can be administered in either order or simultaneously.

Suitable routes of administering the antibody compositions (e.g. human monoclonal antibodies, multispecific and bispecific molecules, immunoconjugates or vaccines) of the invention in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

Detection Methods

In one embodiment, the antibodies (e.g., human monoclonal antibodies, multispecific and bispecific molecules, and compositions) of the invention can be used to detect levels of CD32, or levels of cells which contain CD32 on their membrane surface. In a preferred embodiment, the antibodies of the invention can be used to detect the FcγRIIa-H131 allotype, specifically. Detection of CD32 using an antibody of the invention can be achieved, for example, by contacting a sample (such as an in vitro sample) and a control sample with the anti-CD32 antibody under conditions that allow for the formation of a complex between the antibody and CD32. Any complexes formed between the antibody and CD32 are detected and compared in the sample and the control. For example, standard detection methods, well-known in the art, such as ELISA and flow cytometic assays, can be performed using the compositions of the invention.

Accordingly, in one aspect, the invention further provides methods for detecting the presence of CD32 (e.g., human CD32 antigen) in a sample, or measuring the amount of CD32, comprising contacting the sample, and a control sample, with an antibody of the invention, or an antigen binding portion thereof, which specifically binds to CD32, under conditions that allow for formation of a complex between the antibody or portion thereof and CD32. The formation of a complex is then detected, wherein a difference in complex formation between the sample compared to the control sample is indicative of the presence of CD32 in the sample.

In still another embodiment, the invention provides a method for detecting the presence or quantifying the amount of Fc-expressing cells in vivo or in vitro. The method comprises (i) administering to a subject a composition (e.g., a monoclonal antibody or a multi- or bispecific molecule) of the invention or a fragment thereof, conjugated to a detectable marker; (ii) exposing the subject to a means for detecting said detectable marker to identify areas containing Fc-expressing cells.

The compositions (e.g., human antibodies, multispecific and bispecific molecules) of the invention can also be used to target cells expressing CD32, for example for labeling such cells. For such use, the binding agent can be linked to a molecule that can be detected. Thus, the invention provides methods for localizing ex vivo or in vitro cells expressing CD32. The detectable label can be, e.g., a radioisotope, a fluorescent compound, an enzyme, or an enzyme co-factor.

In a preferred embodiment, the invention provides a method for detecting FcγRIIa-H131 in a sample, comprising:
a) contacting the sample with an antibody, or antigen-binding portion thereof, that binds FcγRIIa-H131 but does not bind FcγRIIa-R131; and
b) detecting the antibody, or antigen-binding portion thereof, bound to FcγRIIa-H131.

Preferably, the antibody is a human antibody of the invention thereof, such as the MDE-9 antibody, which comprises a heavy chain variable region comprising the amino acid sequence of SEQ ID NO: 19; and a light chain variable region comprising the amino acid sequence of SEQ ID NO: 20. Alternatively, the antibody can comprise the VH CDRs of MDE-9, as shown in SEQ ID NOs: 13, 14 and 15, and/or the VL CDRs of MDE-9, as shown in SEQ ID NOs: 16, 17 and 18.

The sample can be, for example, blood cells from a subject or a tissue sample from the subject.

Binding of the antibody can be detected by methods known in the art, such as by flow cytometry or by immunohistochemistry. Suitable detection methods are described in detail in the Examples.

In one embodiment, the method further comprises contacting the sample with an antibody, or antigen-binding portion thereof, that binds FcγRIIa-R131 but does not bind FcγRIIa-H131, which allows for discrimination between expression of FcγRIIa-R131 and FcγRIIa-H131. An example of an antibody that binds FcγRIIa-R131 but does not bind FcγRIIa-H131 is 41H16, described further in the Examples.

The FcγRIIa-R131 and FcγRIIa-H131 polymorphism has been linked to induction of side effects with therapeutic antibodies (Tax et al. (1997) Transplantation 63:106-112) and clinical efficacy of antibodies such as Rituxan® (Weng and Levy (2003) J. Clin. Oncol. 21:3940-3947). Accordingly, the methods of the invention for detecting FcγRIIa-H131 can be used in conjunction with treatment with a therapeutic antibody, to assess or predict side effects and/or clinical efficacy.

Uses of Anti-CD32 Antibodies

The antibodies can be used to inhibit or block CD32 function which, in turn, can be linked to the prevention or amelioration of certain disease symptoms, thereby implicating CD32 as a mediator of the disease. Differences in CD32 expression during a disease state as compared to a non-disease state can be determined by contacting a test sample from a subject suffering from the disease and a control sample with the anti-CD32 antibody under conditions that allow for the formation of a complex between the antibody and CD32. Any complexes formed between the antibody and CD32 are detected and compared in the sample and the control.

In one embodiment, human antibodies, or binding portions thereof, of the present invention can be used to modulate CD32 levels on effector cells, such as by capping and eliminating receptors on the cell surface. Mixtures of anti-Fc receptor antibodies can also be used for this purpose.

In a preferred embodiment, anti-CD32 antibodies can be used to treat autoimmune hemolytic anemia and other cytopenic disorders. In autoimmune hemolytic anemia (AIHA), antibodies against erythrocyte membrane antigens are present, leading to decreased survival of red blood cells (RBC). AIHA is frequently observed after allogeneous bone marrow transplantation due to allo-antibodies to ABO or minor RBC antigens (Drobyski W R et al. (1996) Bone Marrow Transplant 17:1093-1099; Hashimoto C (1998) Clin Rev Allergy Immunol. 16:285-295). In these cases, the response to conventional treatment is generally unsatisfactory, and prolonged courses of immunosuppressive therapy with corticosteroids, might influence engraftment and increase the risk for viral infections. FcγRII has been implicated in AIHA and other auto-immune cytopenic diseases in mice and man and FcγRIIa has been shown to play a role in the clearance of immune-complexes, like human IgG-coated red blood cells in man (Clynes R and Ravetch J V (1995) Immunity 3:21-26; Kumpel B M and Hadley A G (1990) Mol. Immunol. 27:247-256; Dijstelbloem H M et al. (2000) Arthritis Rheum. 43:2793-2800). As described in the Examples, antibodies of the invention can inhibit AIHA in an animal model. Accordingly, another aspect of the invention pertains to a method of treating or preventing autoimmune hemolytic anemia (AIHA) in a subject comprising administering to the subject the antibody, or antigen-binding portion thereof, of the invention such that the subject is treated for AIHA.

In another preferred embodiment, anti-CD32 antibodies can be used to treat immune thrombocytopenia purpura (ITP). ITP is an autoimmune disease characterized by autoantibody-mediated destruction of IgG associated platelets (Crow A R and Lazarus A H (2003) Jpediatr Hematol Oncol 25 Suppl 1:S14-18). Anti-CD32 antibodies bind Fc-gamma receptor, and block the Fc-gamma receptor mediated phagocytosis, prolonging the lifespan of platelets by inhibiting binding of platelets to monocytes (Wallace P K et al. (1997) Cancer Immunol Immunother 45:137-41; Wiener E et al. (2000) Eur JHaematol 65:399-406).

Uses of Bispecific and Multispecific Reagents

Further within the scope of the invention are methods for treating a disorder, such as an autoimmune disorder, a cancer, or a pathogenic infection with the bispecific and multispecific human antibodies described above. Such bispecific and multispecific molecules include at least one binding specificity for CD32 (e.g., a human anti-CD32 antibody of the present invention) and at least one binding specificity for a target antigen. In another embodiment, the antibody includes a third binding specificity for an antigen binding region to a different epitope of the same target antigen and/or receptor. Methods for eliminating unwanted cells, i.e., target cells, or antigen in a subject includes treating the subject with the bispecific or multispecific molecules of the invention. In one embodiment, such methods include administering a bispecific or multispecific molecule of the invention to a subject in which removal of target cells is desired (eg. a tumor bearing subject). In another embodiment, such methods include obtaining an aliquot of a sample of blood or blood cells from a subject, treating the blood or blood cells ex vivo with a therapeutically effective dose of a bispecific or multispecific antibody of the invention in a pharmaceutically acceptable carrier, and returning the treated blood or blood cells to the subject. Preferably, the cells of the sample of blood are isolated and expanded in culture.

Target-specific effector cells, e.g., effector cells linked to compositions (e.g., human antibodies, multispecific and bispecific molecules) of the invention can also be used as therapeutic agents. Effector cells for targeting can be human leukocytes such as macrophages, neutrophils or monocytes. If desired, effector cells can be obtained from the subject to be treated. The target-specific effector cells, can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing the target of interest, and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) of the invention and/or effector cells armed with these compositions can be used in conjunction with chemotherapy. Additionally, combination immunotherapy may be used to direct two distinct cytotoxic effector populations toward tumor cell rejection.

Bispecific and multispecific molecules of the invention can also be used to modulate FcγRIIa levels on effector cells, such as by capping and elimination of receptors on the cell surface. Mixtures of anti-Fc receptors can also be used for this purpose.

The compositions (e.g., human antibodies, multispecific and bispecific molecules and immunoconjugates) of the invention which have complement binding sites, such as portions from IgG1, -2, or -3 or IgM which bind complement, can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

The compositions (e.g., human antibodies, multispecific and bispecific molecules and immunoconjugates) of the invention can also be administered together with complement. Accordingly, within the scope of the invention are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of the invention and the complement or serum can be administered separately.

Use of Immunoconjugates and Combination Therapy

As previously described, human anti-CD32 antibodies of the invention can be co-administered with one or other more therapeutic agents, e.g., an cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy or radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/ml dose once every four weeks and adriamycin is intravenously administered as a 60-75 mg/ml dose once every 21 days.

In one embodiment, immunoconjugates of the invention can be used to target compounds (e.g., therapeutic agents, labels, cytotoxins, radiotoxins immunosuppressants, etc.) to cells which have CD32 cell surface receptors by linking such compounds to the antibody. Thus, the invention also provides methods for localizing ex vivo or in vitro cells expressing CD32 (e.g., with a detectable label, such as a radioisotope, a fluorescent compound, an enzyme, or an enzyme co-factor). Alternatively, the immunoconjugates can be used to kill cells which have CD32 cell surface receptors by targeting cytotoxins or radiotoxins to CD32, such as to CD32-expressing tumor cells to thereby eliminate the tumor cell, or to CD32-expressing antigen-presenting cells to thereby eliminate the APCs as a means to inhibit immune responses (e.g., in autoimmune disorders).

In other embodiments, the subject can be additionally treated with an agent that modulates, e.g., enhances or inhibits, the expression or activity of FcγRIIa eceptors by, for example, treating the subject with a cytokine.

In another embodiment, the subject can be additionally treated with a lymphokine preparation. Cancer cells which do not highly express CD32 can be induced to do so using lymphokine preparations. Lymphokine preparations can cause a more homogeneous expression of CD32 among cells of a tumor which can lead to a more effective therapy. Lymphokine preparations suitable for administration include interferon-gamma, tumor necrosis factor, and combinations thereof. These can be administered intravenously. Suitable dosages of lymphokine are 10,000 to 1,000,000 units/patient.

Use of Vaccines

In a particular embodiment, the invention provides methods for stimulating an immune response against an antigen of interest by immunizing a subject against the antigen, such as a cancer antigen, an antigen found on a pathogen or a cell infected by a pathogen, using a vaccine composition of the invention. Such methods include administering to the subject in a pharmaceutically acceptable carrier a composition comprising a bispecific or multispecific antibody having a binding specificity for CD32 and a binding specificity for an epitope of a pathogenic infectious organism, or of an antigen of an infected cell, or of a cancer cell, whereby the antigen is complexed to the bispecific molecule such that it is targeted to CD32-expressing APCs. Alternatively, the vaccine composition can comprise an anti-CD32 antibody linked to one or more antigens of interest, such as an antigen of a pathogenic infectious organism, or an antigen of infected cells, or an antigen of a cancer cell. The vaccine compositions of the invention target the antigen to antigen presenting cells, thus increasing antigen presentation in order to promote an immune response against the antigen.

Treatment of Autoimmune Diseases

The compositions can be used in vitro or in vivo to treat diseases mediated by or involving CD32, for example, diseases characterized by expression, typically overexpression, of CD32 such as autoimmune disease, including those with a combination of both humoral and cellular autoimmunity, transplantation rejection, or Graft versus Host Disease (GVHD). In one embodiment, the antibodies of the present invention may block the binding site of the natural ligand, IgG, to CD32, such that binding would decrease or prevent the binding of autoantibodies against self-antigens, thereby preventing phagocytosis of the target cell, for example, platelets in idiopathic thrombocytopenic purpura or red blood cells in anemia. The compositions can also be used to treat any diseases mediated by CD32 expressing cells, including CD32 expressing malignancies, e.g., acute leukemia, or any autoimmune diseases mediated by macrophages, activated neutrophils, dendritic cells or NK cells. Examples of such diseases include, but are not limited to, autoimmune hemolytic anemia (AIHA), rheumatoid arthritis (RA), systemic lupus erythematosus (SLE), Systemic Sclerosis, Atopic Dermatitis, Graves' disease, Hashimoto's thyroiditis, Wegner's granulomatosis, Omen's syndrome, chronic renal failure, idiopathic thrombocytopenic purpura (ITP), inflammatory bowel disease (IBD; including Crohn's Disease, Ulcerative Colitis and Celiac's Disease), insulin dependent diabetes mellitus (IDDM), acute infectious mononucleosis, HIV, herpes virus associated diseases, multiple sclerosis (MS), hemolytic anemia, thyroiditis, stiff man syndrome, pemphigus vulgaris and myasthenia gravis (MG).

Treatment of Cancer

In another embodiment, the present invention provides a method for treating or preventing a tumorigenic disorder mediated by or involving human CD32, e.g., Hodgkin's disease, non-Hodgkin's lymphoma, Burkitt's lymphoma, anaplastic large-cell lymphomas (ALCL), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other B-cell lymphomas. The method involves administering to a subject a antibody composition of the present invention in an amount effective to treat or prevent the disorder. The antibody composition can be administered alone or along with another therapeutic agent, such as a cytotoxic or a radiotoxic agent which acts in conjunction with or synergistically with the antibody composition to treat or prevent the CD32 mediated disease.

Kits

Also within the scope of the invention are kits comprising the compositions (e.g., antibodies, human antibodies, immunoconjugates, bispecific molecules, and vaccine conjugates) of the invention and instructions for use. The kit can further contain one or more additional reagents, such as an immunosuppressive reagent, a cytotoxic agent or a radiotoxic agent, or one or more additional human antibodies of the invention (e.g., a human antibody having a complementary activity which binds to an epitope in the CD32 antigen distinct from the first human antibody). Kits typically include a label indicating the intended use of the contents of the kit. The term label includes any writing, or recorded material supplied on or with the kit, or which otherwise accompanies the kit.

The present invention is further illustrated by the following examples which should not be construed as further limiting. The contents of all figures and all references, patents and published patent applications cited throughout this application are expressly incorporated herein by reference.

### EXAMPLES

### Example 1

### Generation of Human Monoclonal Antibodies Against CD32

Antigen

A fusion protein comprised of FcγRIIa-H131 (FcγRIIa with an arginine (R) to histidine (H) substitution residue 131) conjugated to human serum albumin was used as antigen for immunization. In addition, IIA1.6 cells (mouse B cell lymphoma Fc gamma receptor negative cell line) transfected with either FcγRIIa-H131 or FcγRIIa-R131 in PBS were also used for subsequent immunizations.

Transgenic HuMab Mice

Fully human monoclonal antibodies to human CD32 were prepared using the HCo7 strain of HuMab transgenic mice, which expresses human antibody genes. In this mouse strain, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al. (1996) Nature Biotechnology 14:845-851, and a human heavy chain transgene, HCo7, as described in U.S. Pat. Nos. 5,545,806; 5,625,825; and 5,545,807.

HuMab Immunizations:

To generate fully human monoclonal antibodies to FcγRIIa, HuMab mice were immunized intraperitoneally with 50 μg FcγRIIa-H131 conjugated to human serum albumin in complete Freund's adjuvant, and several times with IIA1.6 cells transfected with either FcγRIIa-H131 or FcγRIIa-R131 in PBS. General immunization schemes for HuMab mice are described in Lonberg, N. et al (1994) Nature 368(6474): 856-859; Fishwild, D. et al. (1996) Nature Biotechnology 14: 845-851 and PCT Publication WO 98/24884. The plasma was screened for antibody titers and mice with sufficient titers of anti-CD32 human immunoglobulin were used for fusions.

Generation of Hybridomas Producing Human Monoclonal Antibodies to CD32:

Mice with positive antibody titers were sacrificed, and splenocytes were fused with SP2/0 myeloma cells according to standard laboratory protocols. Resulting hybridomas were screened by enzyme-linked immunosorbent assay (ELISA) for human IgG, K antibodies, and in flow-cytometric assays with IIA1.6 FcγRIIa-R131 and IIA1.6 FcγRIIa-H131 cells. Hybridomas producing human K CD32 antibodies were subcloned by at least two rounds of limiting dilution. Select human antibodies were purified by affinity chromatography, using Sepharose-coupled protein A (Pharmacia, Uppsala, Sweden). Fractions were analyzed by electrophoresis on 4-15% SDS gradient gels, and stained with Coomassie Brilliant Blue. Protein concentrations were determined by optical densitometry at 280 nm, and a PIERCE assay (Rockford, Ill.). F(ab′)₂ fragments were generated by standard methods of digestion of whole antibody, using pepsin and citric acid (Coligan et al., Current Protocols in Immunology: Wiley & Sons, 2002). The preparations were depleted of residual Fc portions by protein A adsorption chromatography. F(ab′)₂ fragments were purified by gel filtration, and checked by 10% SDS-PAGE.

Seven clones were produced. Hybridoma clones MDE-8 and MDE-9 were selected for further analysis.

### Example 2

### Structural Characterization of Human Anti-CD32 Monoclonal Antibodies

The cDNA sequences encoding the heavy and light chain variable regions of the MDE-8 or MDE-9 monoclonal antibodies were obtained from the MDE-8 and MDE-9 hybridomas using standard PCR techniques and were sequenced using standard DNA sequencing techniques.

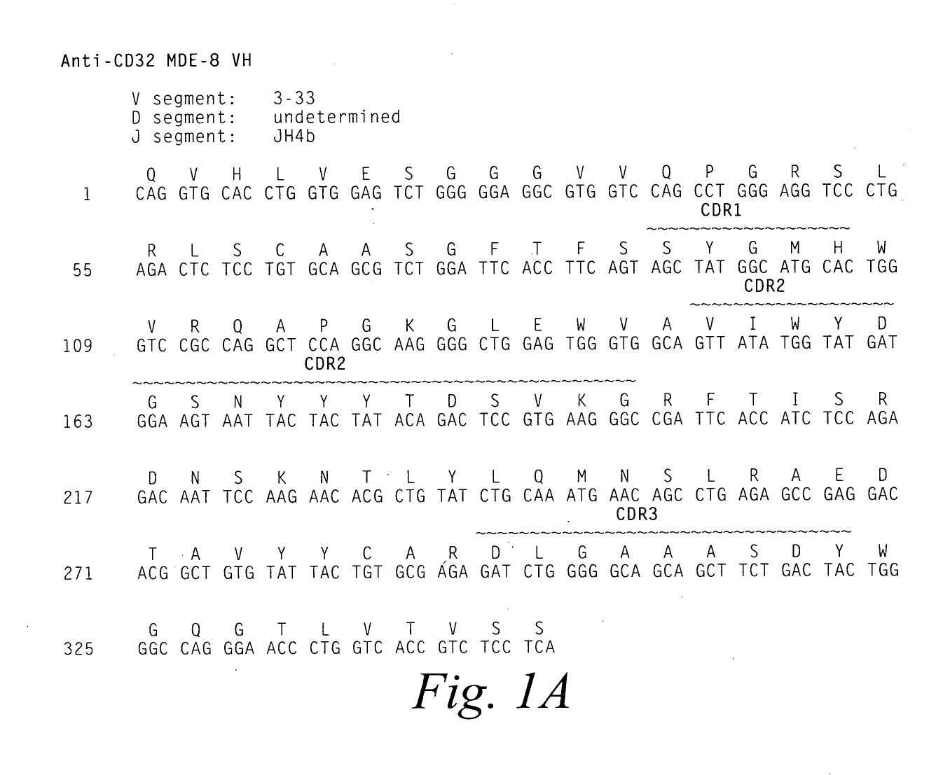

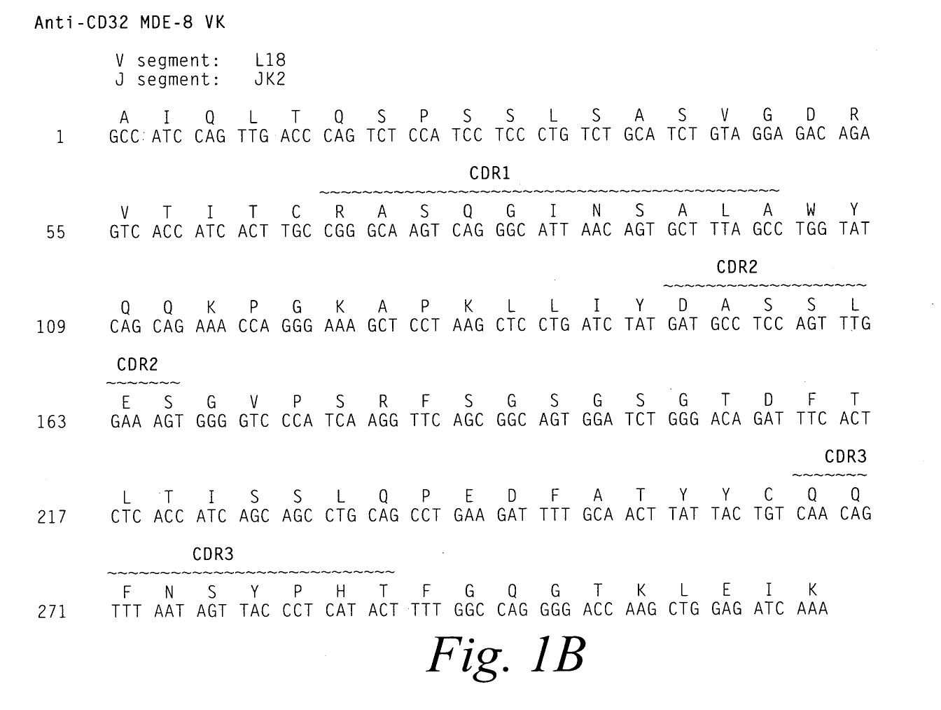

The nucleotide and amino acid sequences of the heavy chain variable region of MDE-8 are shown in FIG. 1A and in SEQ ID NO: 9 and 7, respectively.

The nucleotide and amino acid sequences of the light chain variable region of MDE-8 are shown in FIG. 1B and in SEQ ID NO: 10 and 8, respectively.

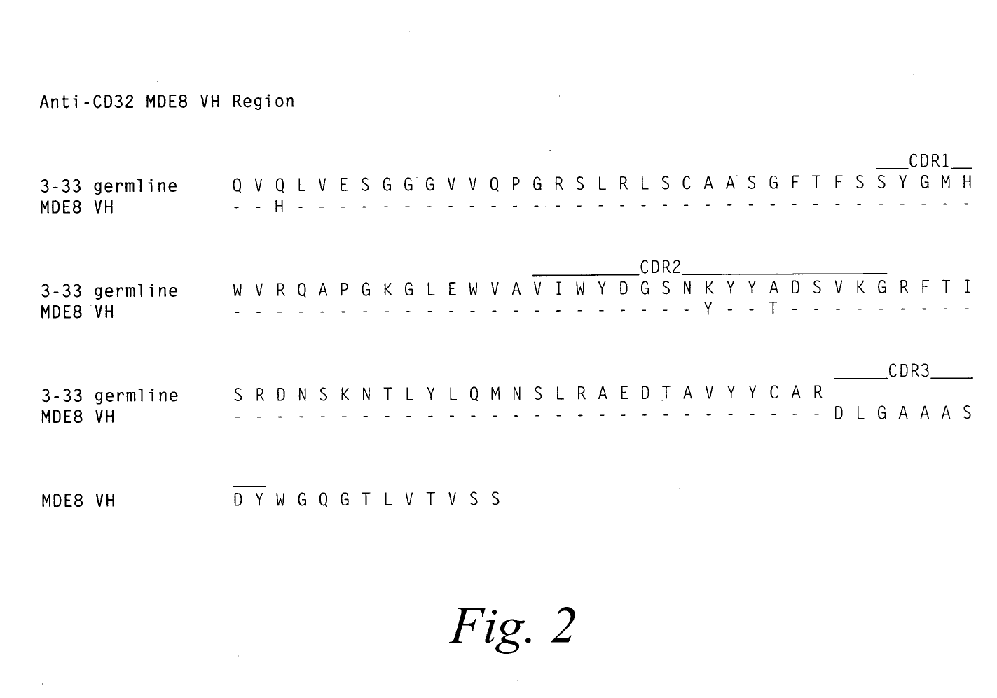

Comparison of the MDE-8 heavy chain immunoglobulin sequence to the known human germline immunoglobulin heavy chain sequences demonstrated that the MDE-8 heavy chain utilizes a VH segment from human germline V_{H} 3-33, an undetermined D segment, and a JH segment from human germline JH4b. The alignment of the MDE-8 VH sequence to the germline V_{H} 3-33 sequence is shown in FIG. 2. Further analysis of the MDE-8 VH sequence using the Kabat system of CDR region determination led to the delineation of the heavy chain CDR1, CDR2 and CD3 regions as shown in FIGS. 1A and 2, and in SEQ ID NOs: 1, 2 and 3, respectively.

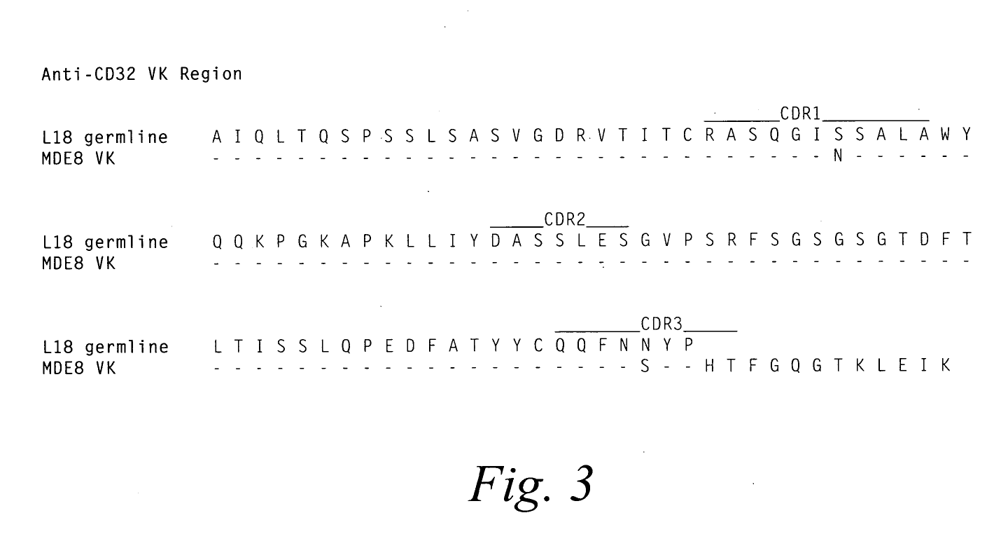

Comparison of the MDE-8 light chain immunoglobulin sequence to the known human germline immunoglobulin light chain sequences demonstrated that the MDE-8 light chain utilizes a VL segment from human germline V_{K} L18 and a JK segment from human germline JK2. The alignment of the MDE-8 Vk sequence to the germline Vk L18 sequence is shown in FIG. 3. Further analysis of the MDE-8 Vk sequence using the Kabat system of CDR region determination led to the delineation of the light chain CDR1, CDR2 and CD3 regions as shown in FIGS. 1B and 3, and in SEQ ID NOs: 4, 5 and 6, respectively.

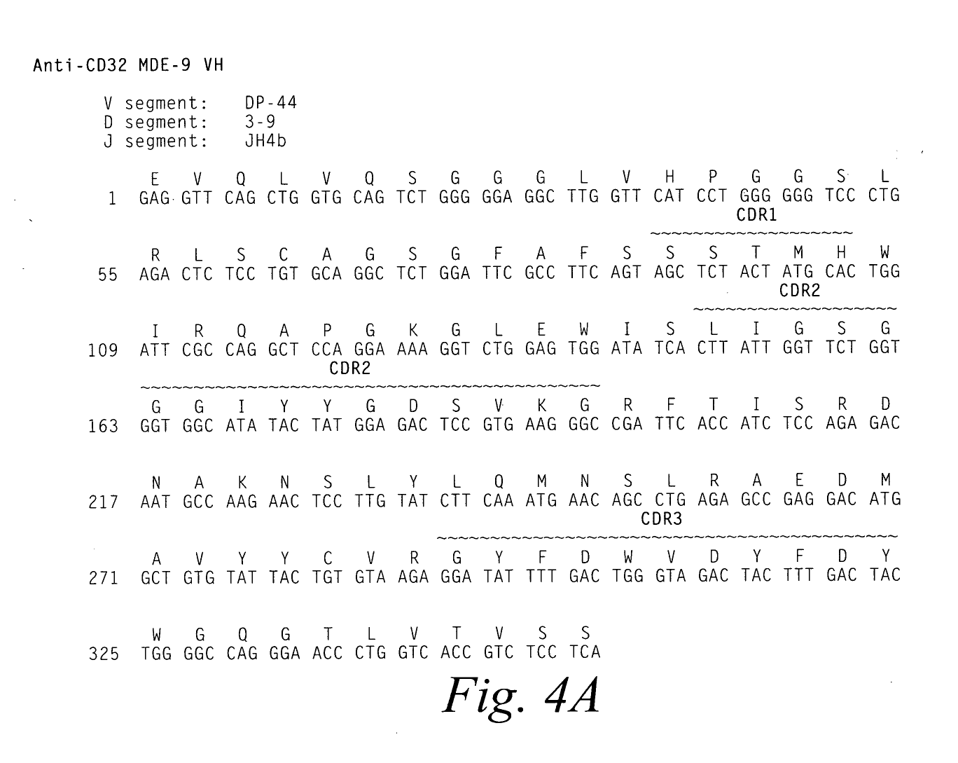

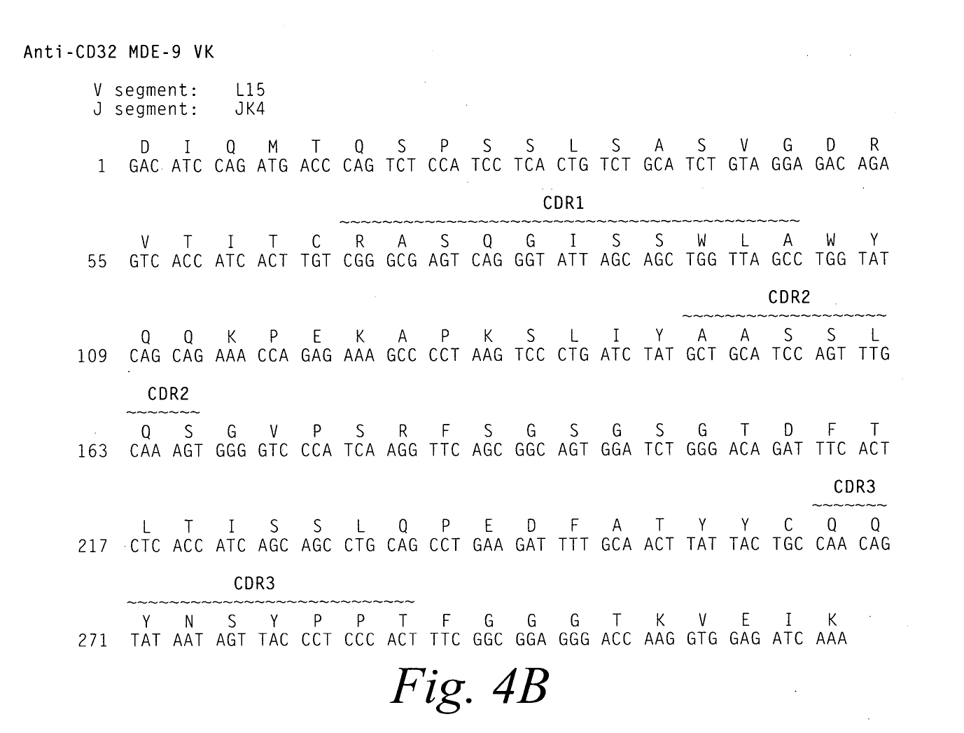

The nucleotide and amino acid sequences of the heavy chain variable region of MDE-9 are shown in FIG. 4A and in SEQ ID NO: 21 and 19, respectively.

The nucleotide and amino acid sequences of the light chain variable region of MDE-9 are shown in FIG. 4B and in SEQ ID NO: 22 and 20, respectively.

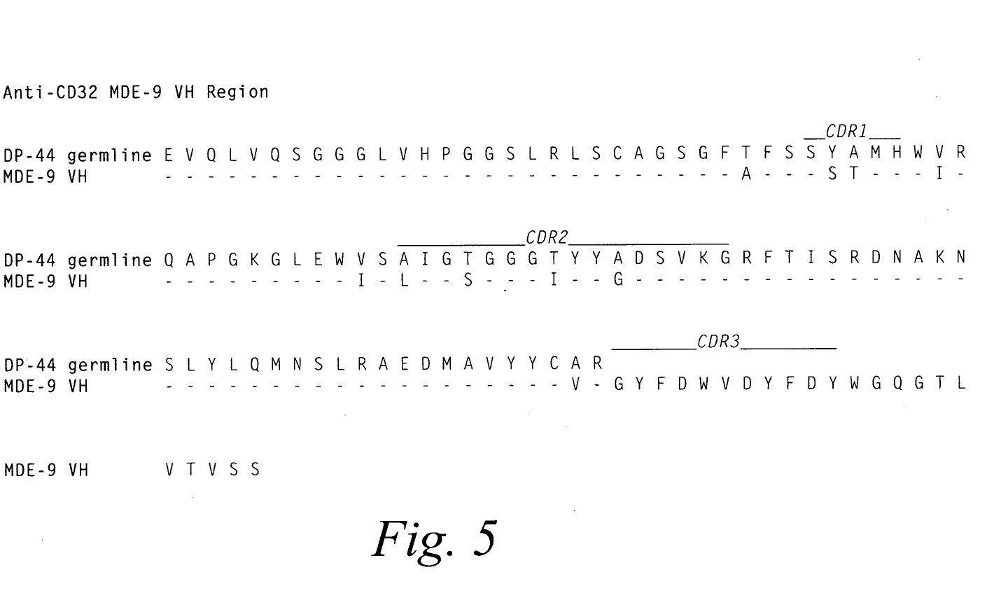

Comparison of the MDE-9 heavy chain immunoglobulin sequence to the known human germline immunoglobulin heavy chain sequences demonstrated that the MDE-9 heavy chain utilizes a VH segment from human germline VH DP-44, a D segment from the 3-9 germline, and a JH segment from human germline JH4b. The alignment of the MDE-9 VH sequence to the germline VH DP-44 sequence is shown in FIG. 5.

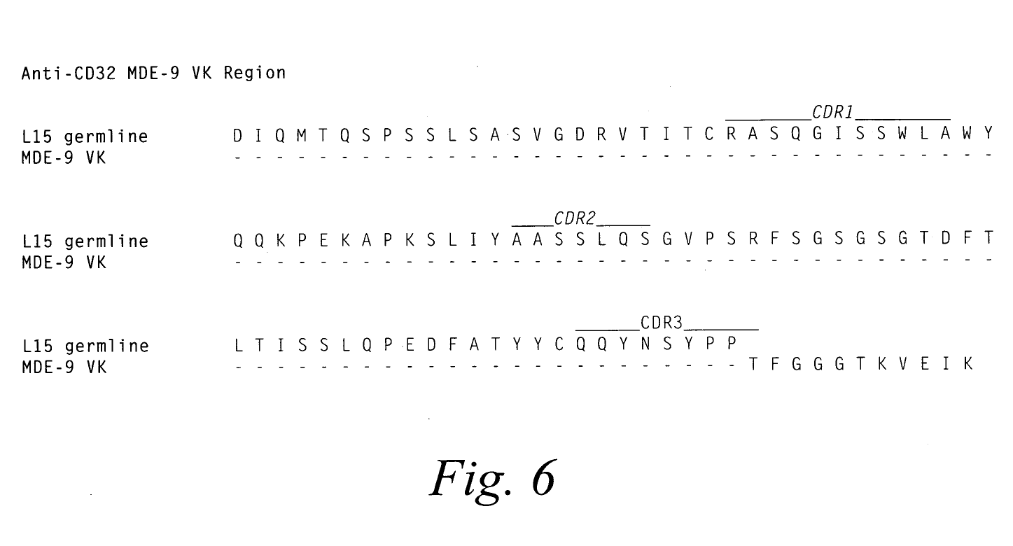

Comparison of the MDE-9 light chain immunoglobulin sequence to the known human germline immunoglobulin light chain sequences demonstrated that the MDE-9 light chain utilizes a Vk segment from human germline Vk L15 and a Jk segment from human germline JK4. The alignment of the MDE-9 Vk sequence to the germline Vk L15 sequence is shown in FIG. 6.

### Example 3

### Binding Characterization of MDE-8

In this example, and/or in Examples 4 and 5, the following materials were used:

Cells

IIA1.6 cells transfected with FcγRIIa-R131 (Van Den Herik-Oudijk et al. (1994) J. Immunol. 152:574-585), Ila-H131 (Van Den Herik-Oudijk (1994), supra), Fca receptor (Morton et al. (1995) J. Biol. Chem. 270:29781-29787), Jurkat cells, naturally expressing FcγRIIIa, IIA1.6 cells expressing FcγRIIb1* (Van Den Herik-Oudijk (1994), supra), as well as Raji cells, expressing CD20 were cultured in RPMI 1640 medium supplemented with 10% heat-inactivated fetal calf serum (FCS, Hyclone, Logan, Utah) and penicilline/streptomycine. The human monocytic cell-line THP-1 (American Type Culture Collection, Rockville, Md.) was cultured in RPMI 1640 medium (GibcoBRL, Grand Island, N.Y.) with 10% FCS and penicilline/streptomycine. Mononuclear cells from healthy donors, allotyped for FcγRIIa by PCR (Carlsson et al. (1998) Blood 92:1526-1531), were isolated from heparinized venous blood using Ficoll-Histopaque (Sigma, St. Louis, Mo.) density gradient centrifugation. Isolated cells were washed twice and resuspended either in RPMI 160 medium with 10% FCS, or phosphate-buffered saline containing 1% bovine serum albumin and 0.02% sodium azide (=immunofluorescence buffer, IFB).

Monoclonal Antibodies

Monoclonal antibodies directed to FcγRI (CD64), mAb 22 (mIgG1), mAb 32 (mIgG1) and mAb 197 (mIgG2a), as well as anti-FcγRIIa mAb IV.3 (mIgG2b) were from Medarex (Annandale, N.J.). Anti-FcγRIIa (CD32) mAb Fli8.26 (mIgG1) was obtained from Research Diagnostics (Flanders, N.J.). Antibody Fli8.26 and mAb 32 were both used as FITC-labeled IgG in direct immuno fluorescent staining, and mAb 197 was used as unlabeled IgG to block FcγRI in select experiments (Pfefferkorn et al. (1989) J. Biol. Chem. 264:14112-14120). The anti-CD20 mAb B1 (Coulter Corporation, Miami, Fla.) and Fc receptor antibodies Gran-1, against CD16 (Sanquin, Amsterdam, Netherlands), and A77 against CD89 (Medarex) were used as positive controls in binding studies. mAb 14.1 (human IgG1, anti-FcαRI) was used as isotype control (Van Spriel et al. (2002) J. Immunol. 169:3831-3836). Mouse IgG1 anti-murine erythrocyte antibody 105-2H was kindly provided by Dr. Izui (Dept. of Pathology, University of Geneva, Geneva, Switzerland) and is described in de Sa Oliveira et al. (1996) J. Clin. Exp. Immunol. 105:313-320.

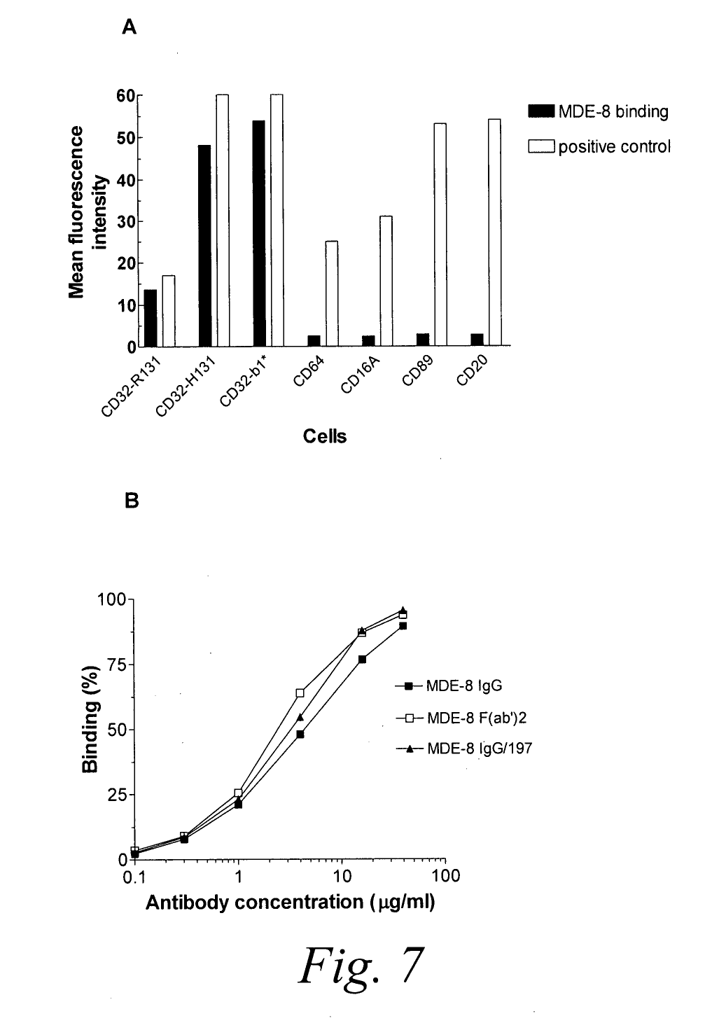

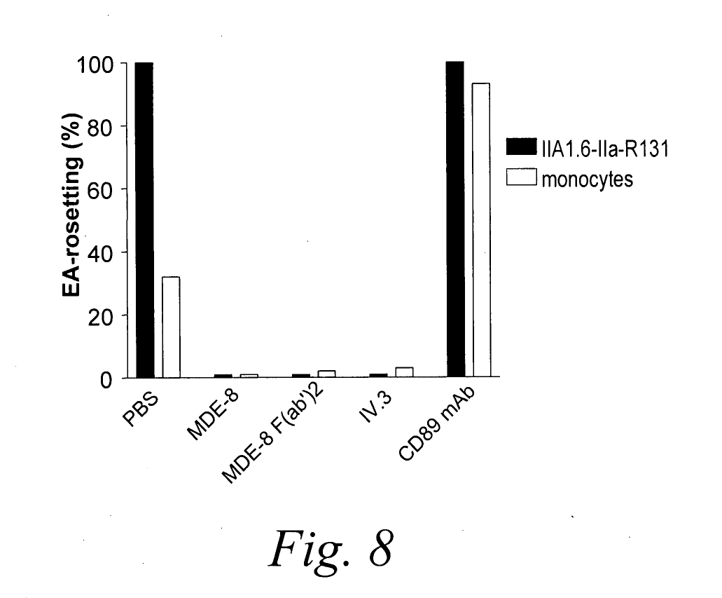

The binding specificity of antibody MDE-8 was examined by incubating the antibody with IIA1.6 cells transfected to express either FcγRIIa-H131, FcγRIIa-R131 or FcγRIIb1*, FcγRI (CD64), FcγRIIIa (CD16) or FcαR (CD89). MDE-8 bound to IIA1.6 cells transfected with FcγRIIa-H131, FcγRIIa-R131 or FcγRIIb1*. No binding was observed to IIA1.6 cells expressing FcγRI (CD64), IIIa (CD16) or FcαR (CD89). These results are shown in FIG. 7A. Furthermore, MDE-8 bound to human peripheral blood monocytes, PMN, platelets and B-cells.

We next assessed whether MDE-8 bound FcγRII via its F(ab) part and studied the binding of purified F(ab′)₂ fragments to THP-1 cells, expressing both FcγRI and IIa. Assays were performed on ice in 96-well, round-bottomed polypropylene microtiter plates (Nunc, Roskilde, Denmark). One hundred thousand THP-1 cells were seeded in 100 μl IFB. Cells were incubated 30 min at 4° C. with MDE-8, with or without pre-incubation with an FcγRI-blocking mAb 197 (Pfefferkorn et al. (1989) J. Biol. Chem. 264:14112-14120), or with MDE-8 F(ab′)₂ fragments. Cells were washed twice, and incubated with FITC-labeled goat F(ab′)₂ anti-human-kappa serum (Jackson, West Grove, Pa.). Flow cytometric analyses were performed using a FACScan flowcytometer (Becton Dickinson, San Jose, Calif.). The results, shown in FIG. 7B, demonstrated that binding curves of MDE-8 F(ab′)₂ fragments were similar to those obtained with whole IgG. Binding curves to THP-1 cells in the presence of the FcγRI-blocking mAb 197 were also nearly identical. Similar results were obtained using human peripheral blood monocytes. Thus, MDE-8 binds to FcγRII via its F(ab) part.

To determine the binding affinity of MDE-8 for FcγRII, Biosensor analysis was performed. Kinetic analyses were performed with a Biacore 3000 (Biacore, Uppsala, Sweden) with MDE-8 at 40 μg/ml. Affinities were measured by immobilizing mAb HSA-FcγRIIa-H131 in 10 mM phosphate buffer (pH 5.00) to a CM-5 sensor chip according to manufacturer prescription. 10 mM glycine-HCl solution pH 2.5 was used for regeneration. Affinity was analysed with Bia evaluation by 1:1 Langmuir Fit. MAb MDE-8 IgG bound to FcγRIIa with a K_{D} of 9×10⁻⁹.

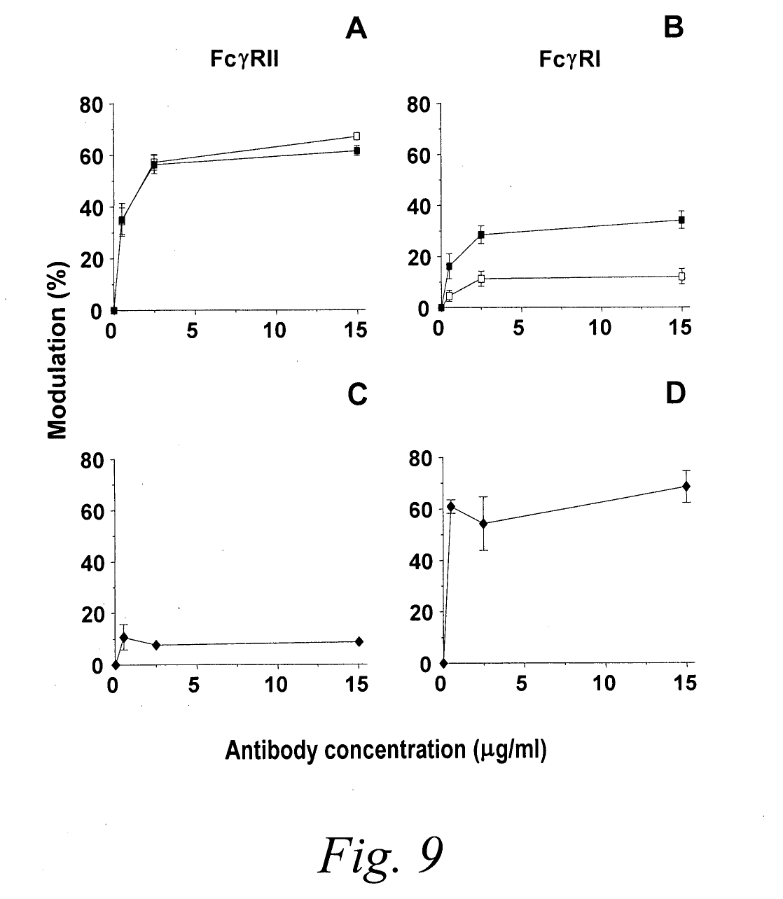

We next assessed the ability of MDE-8 to inhibit EA-rosette formation. Erythrocytes sensitized with a mouse IgG1 anti-glycophorine mAb were used to assay FcγRIIa binding (van de Winkel et al. (1989) Scand. J. Immunol. 29:23-31; van de Winkel et al. (1988) J. Immunol. 140:3515-3521). Monocytes of healthy FcγRIIa-R/R131 donors, or IIA 1.6 FcγRIIa-R131 transfectants incubated with 10 μg/μl MDE-8 IgG or 10 μg/ml MDE-8 F(ab′)₂ fragments (1 h, 4° C.). CD32-blocking mAb IV.3 and hIgG1 CD89 mAb 14.1 (Van Spriel et al. (2002) J. Immunol. 169:3831-3836) were used as controls. Human erythrocytes were opsonized (30 min at 37° C.) with mouse IgG1 anti-glycophorin A mAb, binding selectively to FcγRIIa-R131 (van de Winkel et al. (1989) supra; Braakman et al. (1992) Cell. Immunol. 143:97-107). Monocytes and transfectants were incubated with opsonized erythrocytes in a ratio of 1:5. Cells and erythrocytes were pelleted (10 min at 250×g) and incubated at 4° C. for 1 h. Cells were then resuspended in RPMI 1640 medium. Cells with at least three bound erythrocytes were microscopically scored as EA-rosettes. Formation of EA-rosettes could be blocked by both IgG, and F(ab′)₂ fragments of MDE-8 at similar levels as mAb IV.3, as shown in FIG. 8 A control human IgG1 mAb directed to CD89 IgG1 had no effect on EA-rosette formation

### Example 4

### Effect of MDE-8 on FcR Modulation

The effect of MDE-8 on FcγR expression on THP-1 cells and monocytes was examined. After overnight incubation of THP-1 cells or monocytes with MDE-8, membrane expression of FcγRIIa and FcγRI (CD64) was assayed with directly-labeled monoclonal antibodies, binding outside the ligand binding regions of FcγRIIa, and FcγRI (FLI8.26, and 32.2, respectively) (Pfefferkorn et al. (1989) J. Biol. Chem. 264:14112-14120; van de Winkel et al., Leucocyte Typing. Oxford:Oxford University Press, 1995:823-826).

To conduct the assay, THP-1 cells were grown overnight at 37° C. in RPMI 1640 medium with 10% FCS and recombinant IFN-γ (300 U/ml) (Amgen, Thousand Oaks, Calif.) to enhance FcγRI expression (Guyre et al. (1983) J. Clin. Invest. 72:393-397). Cells were washed twice in RPMI 1640 medium and divided over two tubes. The first suspension was kept at 4° C. in IFB, the second was resuspended in RPMI 1640 medium at 37° C. MDE-8 IgG was added to both sets of cells in different concentrations and incubated overnight. As a control, cells were incubated with the mAb H22, which selectively modulated CD64 (Wallace et al. (1997) J. Leukoc. Biol. 62:469-479). Cells were washed twice with IFB, and kept at 4° C. FITC-labeled mAb FLI8.26 and mAb 32.2, binding to FcγIIa, and FcγRI outside their respective ligand-binding regions, were added at 10 μg/ml to detect FcγRII, and FcγRI expression. Flow cytometric analyses were performed on a FACSscan. Mean fluorescence intensity of cells incubated in IFB at 4° C. was set at 100%. Modulation was expressed as percent decrease in receptor surface expression as in the study by Wallace et al. (1997) J. Leukoc. Biol. 62:469-479.

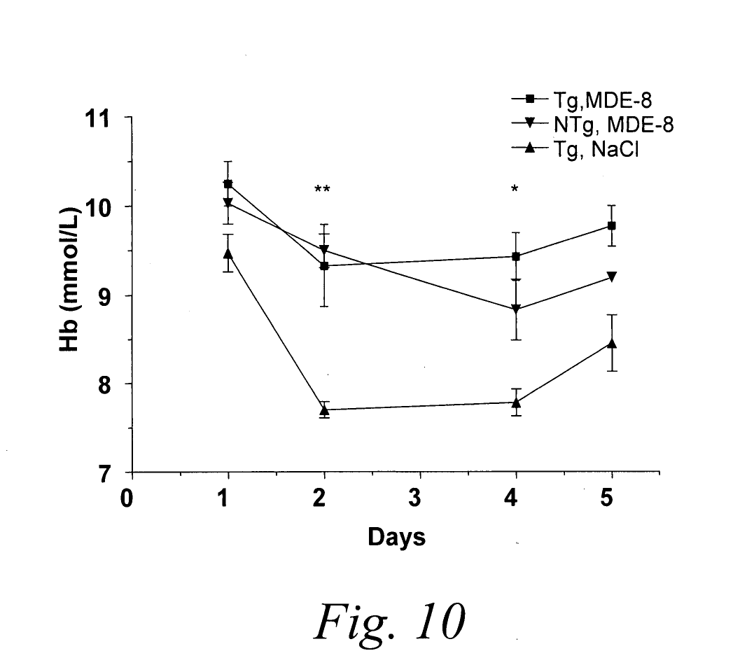

MDE-8 IgG and MDE-8 F(ab′)₂ fragments induced a 60 to 70% decrease of FcγRIIa membrane expression, both on THP-1 cells and human monocytes (FIG. 9A). Notably, MDE-8 IgG induced a ˜30% decrease in membrane expression of FcγRI as well, whereas MDE-8 F(ab′)₂ fragments did not (FIG. 9B). This indicated the Fc-tail of MDE-8 IgG to be responsible for the reduction in FcγRI surface expression. Incubation with CD64 mAb H22 had no effect on FcγRIIa expression (FIG. 9C), but induced a reduction of FcγRI membrane expression of ˜60% (FIG. 9D). These data documented MDE-8's ability to down-modulate both FcγRIIa and FcγRI on phagocytic cells. FIG. 9A-9D shows the results of 7 representative experiments performed on THP-1 cells.

### Example 5

### Prevention of Autoimmune Hemolytic Anemia by MDE-8

The ability of MDE-8 to inhibit autoimmune hemolytic anemia (AIHA) in an animal model was tested. In the model, a human FcγRIIa transgenic mouse was bred into an FcR γ-chain knock-out strain (lacking all murine activatory Fcγ-receptors) (see van Vugt et al. (1996) Blood 87:3593-3599; Park et al. (1998) J. Clin. Invest. 102:1229-1238). The sole activating FcγR in these mice is human FcγRIIa (McKenzie et al. (1999) J. Immunol. 162:4311-4318).

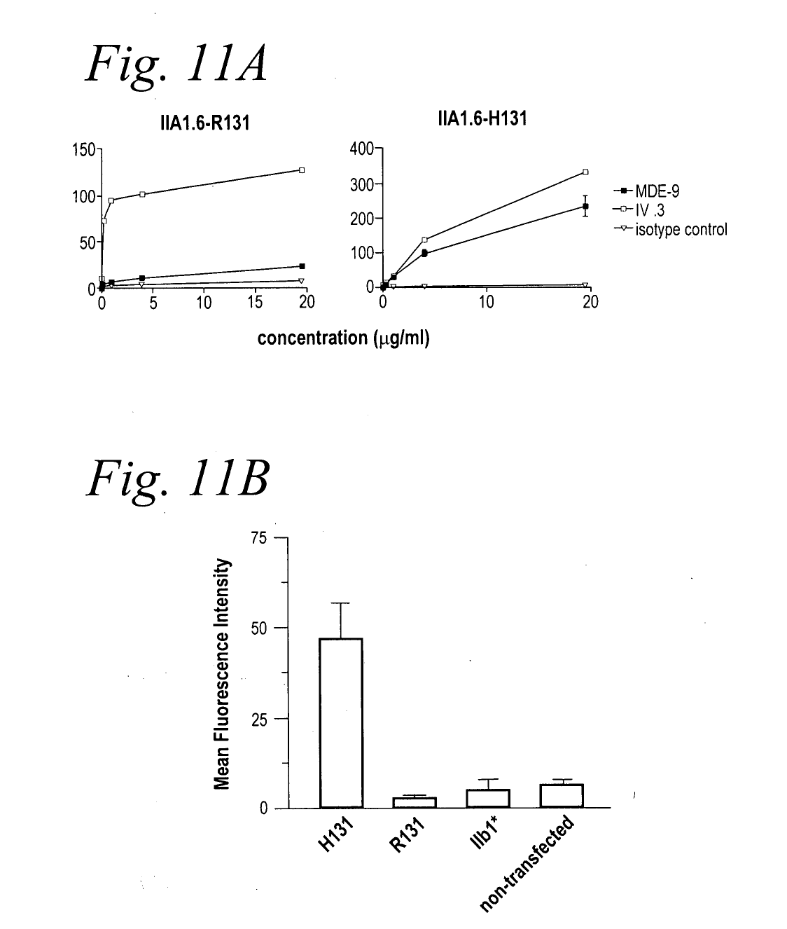

To induce an immune complex-mediated anemia in these mice, the mIgG1 anti-mouse erythrocyte antibody 105.2H (described in Fossati-Jimack et al. (2000) J. Exp. Med. 191:1293-1302) was used. Hemolytic anemia was induced by a single intraperitoneal (IP) injection of 400 μg mIgG1 anti-mouse erythrocyte mAb 105.2H in 8-12 week old female FcγRIIa-Tg mice, and in non-trangenic mice (NTg) in an FcRγ-chain KO background (controls). Hemolytic anemia could effectively be induced in FcγRIIa-Tg mice, but not non-transgenic mice. For FcγRIIa blockade, 5 μg/gram MDE-8 was injected intravenously 60 min prior to IP administration of mAb 105.2H. Control mice were injected with equal volumes of physiological saline (0.9% NaCl). Blood samples were obtained daily (days 0 to 8) from the retroorbital plexus, or tail veins and were collected in heparinized tubes. Erythrocyte counts were measured in whole blood using a Cell-Dyn1700 multiparameter hematology analyser (Abbott, Abbott Park, Ill.). The results, shown in FIG. 10, demonstrate that a single intravenous injection of 80 μg MDE-81 hr prior to the infusion of the mIgG1 mAb 10.2H effectively blocked the induction of hemolytic anemia compared to levels observed in control (NTg) mice.

### Example 6

### Binding Characterization of MDE-9

In this example, and/or in Examples 7, 8 and 9, the following materials and methods were used:

Monoclonal Antibodies

Murine mAb 41 H16 (mIgG2a) recognizing FcγRIIa-R131 and FcγRIIb was a kind gift from Dr. B. Longenecker (Edmonton, Canada) (Gosselin et al. (1990) J. Immunol. 144:1817-1822). FITC-labeled MAb IV.3 (mIgG2b) was provided by Medarex (Annandale, N.J.). Antibody ATI 0, recognizing FcγRIIa and FcγRIIb (van den Herik-Oudijk (1995) Leucocyle Typing V, Oxford University Press, Oxford, pp. 832-835), was from Biosource (Camarillo, Calif.). Antibodies CIKM5 and FLI 8.2 were obtained from the CD32 Cluster Workshop (lerino et al. (1993) J. Immunol. 150:1794-1803; van de Winkel and Anderson (1995) Leucocyte Typing V, Oxford University Press, Oxford, pp. 823-826). MAb 197 (mIgG2a, Medarex) (Vance et al. (1989) J. Immunol. Methods 118:287-296) was used in select experiments to block non-specific Fc-binding of antibodies to the high affinity FcγRI (CD64). CD89 mAb 14.1 (human IgG1, Medarex) was used as isotype control (van Spriel et al. (2002) J. Immunol. 169:3831-3836).

Cells

IIA1.6 cells transfected with FcγRIIa-R131, IIa-H131(van den Herik-Oudijk et al. (1994) J. Immunol. 152:574-585), Fcα receptor I (Morton et al. (1995) J. Biol. Chem. 270:29781-29787), Jurkat cells expressing FcγRJIIa, RMA-s cells expressing FcγRIIb1, as well as mouse myeloma cell line NS/O expressing CD20, were cultured in RPMI 1640 medium supplemented with 10% heat-inactivated fetal calf serum (FCS, Hyclone, Logan, Utah) and penicillin/streptomycin.

Labeling of Antibodies

One milliliter (1 mg/ml) of mAb MDE-9, and 70 μl of 1 M sodium carbohydrate (pH 9.0), were incubated with 200 μl FITC (Pierce, Rockford, Ill.) (1 mg/ml) in 0.25 M sodium carbohydrate and incubated for 2 hours at room temperature. Labeled protein was separated from excess dye by overnight dialysis in phosphate-buffered saline at 4° C.

For biotinylation MDE-9 (1 mg/ml in 1 M Na₂CO₃) was incubated with 1 mg/ml Biotine-X-NHS (Roche, Basel, Switzerland) in dimethylformamide (Sigma, St Louis, Mo.) for 3 hours at room temperature. The solution was dialyzed overnight with PBS (with sodium azide 100 mg/L) at 4° C.

Monoclonal Ab 41H16 (1 mg/ml) in 0.1M Na₂CO₃ was added to Cy-3 bisfunctional reactive dye (Amersham, London, UK) in a final dye/protein molar ratio of 8, and incubated for 30 min at room temperature. Labeled protein was separated from excess dye via Sephadex G-50 chromatography (Pharmacia), using phosphate-buffered saline (+0.1% sodium azide) for elution.

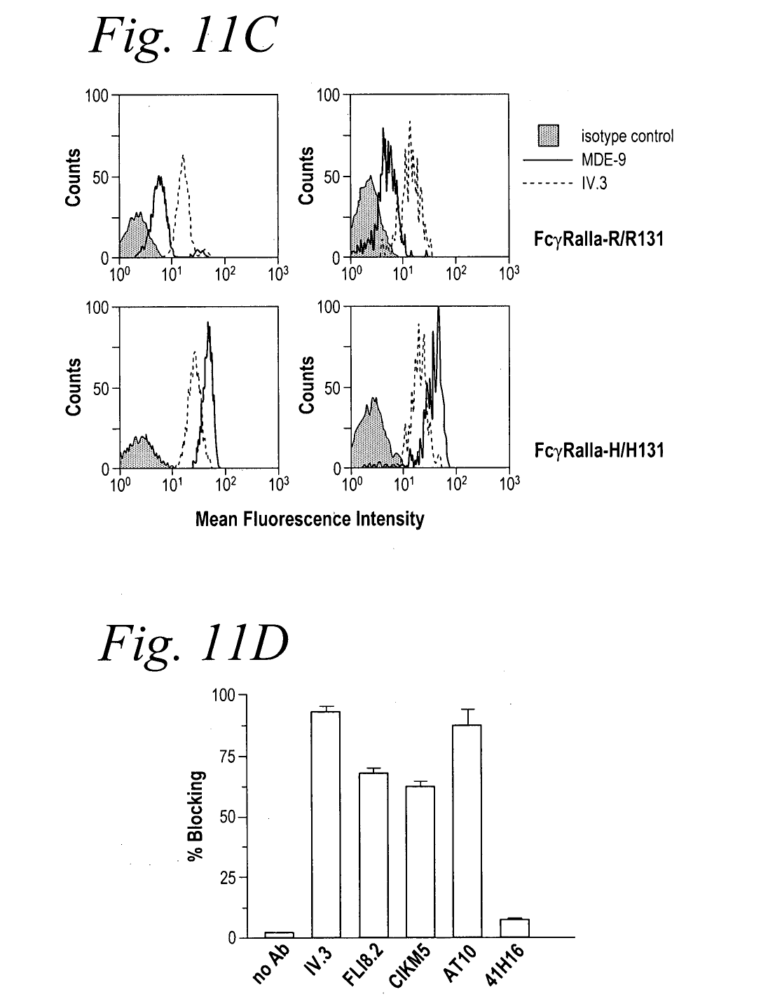

To characterize the binding specificity of antibody MDE-9, binding studies were performed using a panel of IIA1.6 transfectants, as well as isolated PMN and monocytes from FcγRIIa-R/R131 donors. Cells were incubated with different concentrations of mAb MDE-9 for 30 min at 4° C. Cells were washed and resuspendend in phosphate-buffered saline containing 1% BSA and 0.02% sodium azide (=immuno fluorescence buffer, IFB) and FITC-labeled goat anti-human IgG,κ was added for 30 min at 4° C. to detect MDE-9 binding. Cells were washed, resuspended in IFB and analyzed by flow cytometry using a FACSCalibur flow cytometer. As shown in FIGS. 11A and 11B, in lower concentrations (<10 μg/ml) MDE-9 strongly bound to FcγRIIa-H131 transfectants, but not to transfectants expressing FcγRIIa-R131, transfectants expressin FcγRIIb* or non-transfected cells. As shown in FIG. 11C, MDE-9-FITC showed a ˜10-fold binding selectivity for isolated monocytes as well as PMN of FcγRIIa-H/H131 compared to monocytes and PMN of FcγRIIa-R/R131 donors. Residual binding of MDE-9 to FcγRIIa-R/R131 donors might be due to binding to the high affinity Fcγ receptor, FcγRI (CD64), since MDE-9 is a huIgG1 isotype.

To examine the epitope binding of MDE-9, blocking studies were performed using the murine CD32 antibodies IV.3, AT10, FL18.2 and CIKM5. The panel of murine CD32 antibodies was incubated at optimal concentrations for 30 min at 4° C. with IIA1.6-FcγRIIa-H1131 cells. Subsequently, human MDE-9 (˜80% of optimal binding concentration) was added and suspensions were further incubated for 30 min at 4° C. Cells were washed and MDE-9 binding was detected with FITC-labeled goat-anti human IgG1 (Southern Biotechnology, Birmingham, Ala.). Cells were then washed twice, and resuspended in IFB. Flow cytometric analyses were performed using a FACSCalibur flow cytometer (Becton Dickinson, San Jose, Calif.). As shown in FIG. 11D, binding of MDE-9 to FcγRIIa-H131 could be blocked almost completely by pre-incubation of FcγRIIa-transfected cells with CD32 mAb IV.3 and AT10, that both define an epitope in the second Ig-like domain of FcγRIIa. In contrast, CD32 antibodies reactive with the first Ig-like domain (FL18.2, CIKM5) affected MDE-9 binding only partially. 41H16, which does not bind to FcγRIIa-H131, was used as a control and did not block MDE-9 binding.

These data indicate that MDE-9 binds to an epitope within the second Ig-like domain of FcγRIIa that is critically dependent on the presence of a histidine at position 131.

### Example 7

### Immunohistochemistry with MDE-9

The ability of MDE-9 to recognize FcγRIIa-H131 by immunohistochemistry was examined using skin biopsies from participants in a study on graft-versus-host disease upon allogeneic bone marrow transplantation. Skin biopsies of patients who did not develop graft-versus-host disease were used to determine FcγRIIa allotypes via immunohistochemistry using MDE-9. PCR genotyping was used for comparison purposes.

For immunohistochemistry, five μm frozen sections were generated and mounted onto coated slides. After drying overnight, sections were fixed with acetone (at room temperature) and air-dried. Slides were pre-incubated with 10% normal human serum (NHS) and 10% normal goat serum in PBS for 20 min, and subsequently incubated with 5 μg/ml biotinylated MDE-9 in PBS with 1% NHS and 1% normal goat serum for 60 min at room temperature. Next, slides were washed three times with PBS-Tween (0.05%) and incubated for 30 min with Streptavidine-ABC-AP (DAKO, Glostrup, Denmark). Slides were washed again with PBS-Tween (0.05%), and with 0.1 M TRIS/HCl (pH 8.5) for 1 min. Diaminobenzidine (Sigma) was used as substrate, resulting in brown staining. After washing with distilled water, slides were counter-stained with hematoxyllin for 1 min. Subsequently, slides were washed for 10 min in running distilled water, and embedded with aquamount (Thepen et al. (2000) Nat. Biotechnol. 18:48-51).

PCR genotyping of FcγRIIa was performed on genomic DNA isolated from heparinized whole blood samples by QIAamp DNA Minikits (Qiagen, Valencia, Calif.), using polymerase chain reaction (PCR-) based genotyping as described by Carlsson et al. (1998) Blood 92:1526-1531. DNA samples from FcγRIIa-R/R131, IIa-R/H131 and IIa-H/H131 individuals, allotyped by sequencing, were always included as internal controls.

Skin biopsies from allotyped FcγRIIa-H131 homozygous patients (n=2) displayed positive cells upon incubation with MDE-9, whereas FcγRIIa-R131 homozygous patients (n=2) only showed background staining. In heterozygous individuals, the presence of FcγRIIa-H131 molecules was detected. The latter staining, however, was less intense compared to staining of FcγRIIa-H/H131 homozygous skin biopsies (n=3).

These experiments demonstrated that MDE-9 is suitable for use in immunohistochemistry assays for detection of FcγRIIa-H131.

### Example 8

### Single Tube FcγRIIa-Allotyping Using MDE-9

In this example, use of MDE-9 for FcγRIIa allotyping by flow cytometry was evaluated using heparinized whole blood and FITC-labeled MDE-9 and Cy3-labeled 41H16. Heparinized blood was drawn from healthy volunteers (n=15) with known FcγRIIa allotypes (genotyped by PCR and sequencing). Fifty μl whole blood were incubated with 25 μl MDE-9-FITC (final concentration 10 μg/μl) and 25 μl 41H16-Cy3 (final concentration 4 μg/ml) for 30 min at room temperature. FACS lysing solution (Becton Dickinson) was then added for 10 min and cells were washed, resuspended in IFB, and flow cytometric analyses were performed using a FACSCalibur flow cytometer. Dot plot diagrams of FITC or Cy-3 fluorescence intensity of PMN were used for determination of FcγRIIa allotype.

Results of FcγRIIa allotyping by flow cytometry were in complete agreement with those of PCR genotyping in matched experiments. The MDE-9 antibody displayed ˜1.5 times higher mean fluorescence intensities for FcγRIIa-H/H131 individuals, compared to heterozygous individuals, and ˜7-fold higher intensities than for homozygous IIa-R131 individuals. 41H16-Cy3 staining exhibited a reverse pattern, with the highest mean fluorescence in IIa-R/R131 homozygous individuals, and intermediate, and low fluorescence intensities with IIa-R/H131 heterozygous, and IIa-H/H131 homozygous individuals, respectively. Overnight storage of blood samples at room temperature did not affect flow cytometric results.

These experiments demonstrated that MDE-9 is suitable for use in single tube FcγRIIa allotyping by flow cytometry. Thus, provided herein is a rapid assay for flow cytometric determination of FcγRIIa allotypes, combining a FITC-labeled FcγRIIa-H131-recognizing mAb (MDE-9) with a Cy-3 labeled FcγRIIa-R131-recognizing mAb (41H16). Flow cytometric data correlated completely with those generated by PCR-genotyping. Despite the contribution of 41H16 binding to FcγRIIb, the combination with MDE-9 is suitable for allotyping of FcγRIIa.

### Example 9

### Discrimination of FcγRIIa-H131 and FcγRIIb Expression Levels on Monocytes using mAbs MDE-9 and 41H16

In this example, the expression levels of FcγRIIa-H131 and FcγRIIb on peripheral blood mononuclear cells from IIa-H/H131 homozygous individuals was examined, with or without cytokine treatment of the cells, using MDE-9 (which binds FcγRIIa-H131) and 41H16 (which binds FcγRIIb and FcγRIIa-R131, the latter of which is absent in H/H131 homozygous individuals).

Monocytes were purified from PBMC by depletion of non-monocytes using MACS cell sorting according to the manufacturer's instructions (Mylteni Biotec, Bergisch Gladbach, Germany). Monocytes from FcγRIIa-H/H131 homozygous healthy donors (n=7) were incubated for 48 hours either with medium alone, medium with IFN-γ 800 U/ml (Boehringer Ingelheim, Alkmaar, The Netherlands), medium with IL-4 (200 ng/ml), or medium with IL-10 (10 ng/ml; both from Sigma, Saint Louis, Mo.). Monocytes (5×10⁵ cells/well) were incubated at 37° C. (5% CO₂ in humid air) in flat-bottom 24 well micro titer plates in a total volume of 1 ml RPMI 1640 medium (Gibco Invitrogen, Breda, Netherlands) supplemented with 1% penicillin, streptomycin sulphate, and glutamine and 10% human heat-inactivated pooled AB+serum (Red Cross Blood Transfusion Center, Utrecht, The Netherlands). Following culture, monocytes were harvested on ice and stained for FcγRIIb (using mAb 41H116) and FcγRIIa-H131 (using mAb MDE-9). Flow cytometric analyses were performed as described above in Example 8.

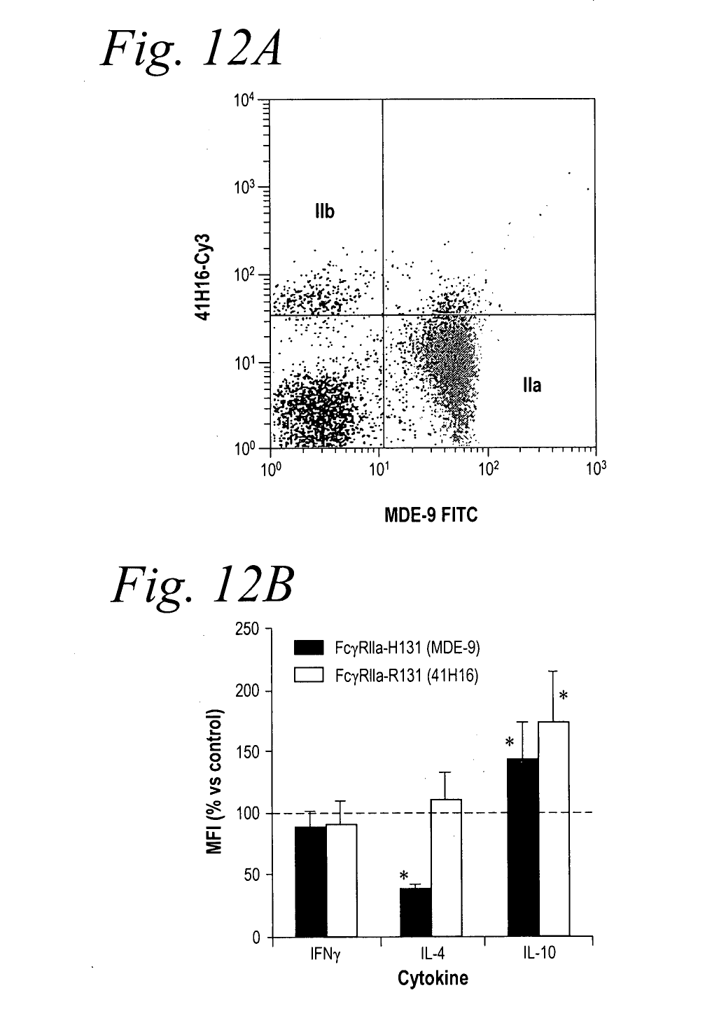

As shown in FIG. 12A, the availability of MDE-9 and 41H116 enabled detection of FcγRIIa-H131 (binding MDE-9-FITC) and FcγRIIb (binding 41H16-Cy3) surface expression on peripheral blood mononuclear cells from IIa-H/H131 homozygous individuals. Differential regulation of FcγRIIa and FcγRIIb on monocytes by cytokines was demonstrated and is summarized in FIG. 12B. FcγRIIa-H131 surface expression was not significantly changed by IFNγ (−12%). IL-4 inhibited (−61%), whereas IL-10 up regulated (+43%) FcγRIIa expression levels significantly, compared to control cultures (n=6). Surface expression of FcγRIIb (41H16) was not changed by 1FNγ or IL-4, but was significantly up regulated by IL-10 (+74%), compared to control cultures.

EQUIVALENTS

Those skilled in the art will recognize, or be able to ascertain using no more than routine experimentation, many equivalents of the specific embodiments of the invention described herein. Such equivalents are intended to be encompassed by the following claims.

### INCORPORATION BY REFERENCE

All patents, pending patent applications and other publications cited herein are hereby incorporated by reference in their entirety.

SUMMARY OF SEQUENCE LISTING

| | |
|---|---|
| SEQ ID NO: | SEQUENCE |

| | |
|---|---|
| 1 | VH CDR1 a.a. MDE-8 |
| 2 | VH CDR2 a.a. MDE-8 |
| 3 | VH CDR3 a.a. MDE-8 |
| 4 | VK CDR1 a.a. MDE-8 |
| 5 | VK CDR2 a.a. MDE-8 |
| 6 | VK CDR3 a.a. MDE-8 |
| 7 | VH a.a. MDE-8 |
| 8 | VK a.a. MDE-8 |
| 9 | VH n.t. MDE-8 |
| 10 | VK n.t. MDE-8 |
| 11 | VH 3-33 germline a.a. |
| 12 | VK L18 germline a.a. |
| 13 | VH CDR1 a.a. MDE-9 |
| 14 | VH CDR2 a.a. MDE-9 |
| 15 | VH CDR3 a.a. MDE-9 |
| 16 | VK CDR1 a.a. MDE-9 |
| 17 | VK CDR2 a.a. MDE-9 |
| 18 | VK CDR3 a.a. MDE-9 |
| 19 | VH a.a. MDE-9 |
| 20 | VK a.a. MDE-9 |
| 21 | VH n.t. MDE-9 |
| 22 | VK n.t. MDE-9 |
| 23 | VH DP-44 germline a.a. |
| 24 | VK L15 germline a.a. |

1-5. (canceled)

6. An isolated monoclonal antibody, or an antigen-binding portion thereof, comprising a heavy chain variable region that is the product of or derived from a human V_{H} 3-33, 3-13 or DP-44 gene and a light chain derived from a V_{K} L18 or L15 gene, wherein the antibody specifically binds to human CD32.

7-10. (canceled)

11. An isolated monoclonal antibody, or antigen binding portion thereof, comprising:
a heavy chain variable region that comprises CDR1, CDR2, and CDR3 sequences; and a light chain variable region that comprises CDR1, CDR2, and CDR3 sequences, wherein:
(a) the heavy chain variable region CDR3 sequence comprises the amino acid sequence of SEQ ID NO: 3 or SEQ ID NO: 15, and conservative modifications thereof,
(b) the light chain variable region CDR3 sequence comprises the amino acid sequence of SEQ ID NO: 6 or SEQ ID NO: 18, and conservative modifications thereof; and
(c) the antibody specifically binds to human CD32.

12. The antibody of claim 11, wherein said antibody exhibits at least one of the following properties:
a) binds FcγRIIa-H131, FcγRIIa-R131, FcγRIIb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.

13. The antibody of claim 11, wherein the heavy chain variable region CDR2 sequence comprises the amino acid sequence of SEQ ID NO: 2 or SEQ ID NO: 14, and conservative modifications thereof; and the light chain variable region CDR2 sequence comprises the amino acid sequence of SEQ ID NO: 5 or SEQ ID NO: 17, and conservative modifications thereof.

14. The antibody of claim 13, wherein the heavy chain variable region CDR1 sequence comprises the amino acid sequence of SEQ ID NO: 1 or SEQ ID NO: 13, and conservative modifications thereof; and the light chain variable region CDR1 sequence comprises the amino acid sequence of SEQ ID NO: 4 or SEQ ID NO: 16, and conservative modifications thereof.

15. The antibody of claim 11, wherein the antibody is selected from the goup consisting of a human antibody a humanized antibody and a chimeric antibody.

16. (canceled)

17. An isolated monoclonal antibody, or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NO: 7 and 19;
(b) the light chain variable region comprises an amino acid sequence that is at least 80% homologous to an amino acid sequence selected from the group consisting of SEQ ID NO: 8 and 20; and
(c) the antibody specifically binds to human CD32.

18. The antibody of claim 17, wherein said antibody exhibits at least one of the following properties:
a) binds FcγRIIa-H131, FcγRIIa-R131, FcγRIlb1*, but does not bind FcγRI (CD64), FcγRIII (CD16) or FcαR (CD89);
b) inhibits FcγRIIa ligand binding;
c) down-modulates surface expression of FcγRIIa;
d) inhibits autoimmune hemolytic anemia; or
e) binds FcγRIIa-H131 but does not bind FcγRIIa-R131 or FcγRIIb1*.

19. The antibody of claim 17, wherein the antibody is selected from the group consisting of a human antibody, a humanized antibody and a chimeric antibody.

20-29. (canceled)

30. A composition comprising the antibody, or antigen-binding portion thereof, of claim 6, and a pharmaceutically acceptable carrier.

31. An immunoconjugate comprising the antibody, or antigen-binding portion thereof, of claim 6, linked to a second agent.

32-35. (canceled)

36. A bispecific or multispecific molecule comprising the antibody, or antigen-binding portion thereof, of claim 6, linked to a second functional moiety having a different binding specificity than said antibody, or antigen binding portion thereof.

37. The bispecific or multispecific molecule of claim 36, wherein the second functional moiety comprises an antibody or a cell receptor ligand.

38-41. (canceled)

42. A composition comprising the bispecific or multispecific molecule of claim 36 and a pharmaceutically acceptable carrier.

43. A vaccine conjugate comprising the antibody, or antigen-binding portion thereof, of claim 6 linked to an antigen.

44-46. (canceled)

47. An isolated nucleic acid molecule encoding the antibody, or antigen-binding portion thereof, of claim 6.

48. An expression vector comprising the nucleic acid molecule of claim 47.

49. A host cell comprising the expression vector of claim 48.

50. A transgenic mouse comprising human immunoglobulin heavy and light chain transgenes, wherein the mouse expresses the antibody of claim 6.

51. A hybridoma which produces the antibody of claim 6.

52-62. (canceled)


SEQUENCE LISTING (24 sequences)

SEQ ID NO: 1 - protein, 5 aa, Homo sapiens
SYGMH

SEQ ID NO: 2 - protein, 17 aa, Homo sapiens
VIWYDGSNYYYTDSVKG

SEQ ID NO: 3 - protein, 9 aa, Homo sapiens
DLGAAASDY

SEQ ID NO: 4 - protein, 11 aa, Homo sapiens
RASQGINSALA

SEQ ID NO: 5 - protein, 7 aa, Homo sapiens
DASSLES

SEQ ID NO: 6 - protein, 9 aa, Homo sapiens
QQFNSYPHT

SEQ ID NO: 7 - protein, 118 aa, Homo sapiens
QVHLVESGGGVVQPGRSLRLSCAASGFTFSSYGMHWVRQAPGKGLEWVAVIWYDGSNYYYTDSVKGRFTISRDNSKNTLYLQMNSLRAEDTAVYYCARDLGAAASDYWGQGTLVTVSS

SEQ ID NO: 8 - protein, 107 aa, Homo sapiens
AIQLTQSPSSLSASVGDRVTITCRASQGINSALAWYQQKPGKAPKLLIYDASSLESGVPSRFSGSGSGTDFTLTISSLQPEDFATYYCQQFNSYPHTFGQGTKLEIK

SEQ ID NO: 9 - DNA, 354 bp, Homo sapiens
  CDS (1)..(354)
caggtgcacctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaattactactatacagactccgtgaagggccgattcaccatctccagagacaattccaagaacacgctgtatctgcaaatgaacagcctgagagccgaggacacggctgtgtattactgtgcgagagatctgggggcagcagcttctgactactggggccagggaaccctggtcaccgtctcctca

SEQ ID NO: 10 - DNA, 321 bp, Homo sapiens
  CDS (1)..(321)
gccatccagttgacccagtctccatcctccctgtctgcatctgtaggagacagagtcaccatcacttgccgggcaagtcagggcattaacagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctcatacttttggccaggggaccaagctggagatcaaa

SEQ ID NO: 11 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 12 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNNYP

SEQ ID NO: 13 - protein, 5 aa, Homo sapiens
SSTMH

SEQ ID NO: 14 - protein, 16 aa, Homo sapiens
LIGSGGGIYYGDSVKG

SEQ ID NO: 15 - protein, 11 aa, Homo sapiens
GYFDWVDYFDY

SEQ ID NO: 16 - protein, 11 aa, Homo sapiens
RASQGISSWLA

SEQ ID NO: 17 - protein, 7 aa, Homo sapiens
AASSLQS

SEQ ID NO: 18 - protein, 9 aa, Homo sapiens
QQYNSYPPT

SEQ ID NO: 19 - protein, 119 aa, Homo sapiens
EVQLVQSGGGLVHPGGSLRLSCAGSGFAFSSSTMHWIRQAPGKGLEWISLIGSGGGIYYGDSVKGRFTISRDNAKNSLYLQMNSLRAEDMAVYYCVRGYFDWVDYFDYWGQGTLVTVSS

SEQ ID NO: 20 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 21 - DNA, 357 bp, Homo sapiens
  CDS (1)..(357)
gaggttcagctggtgcagtctgggggaggcttggttcatcctggggggtccctgagactctcctgtgcaggctctggattcgccttcagtagctctactatgcactggattcgccaggctccaggaaaaggtctggagtggatatcacttattggttctggtggtggcatatactatggagactccgtgaagggccgattcaccatctccagagacaatgccaagaactccttgtatcttcaaatgaacagcctgagagccgaggacatggctgtgtattactgtgtaagaggatattttgactgggtagactactttgactactggggccagggaaccctggtcaccgtctcctca

SEQ ID NO: 22 - DNA, 321 bp, Homo sapiens
  CDS (1)..(321)
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctcccactttcggcggagggaccaaggtggagatcaaa

SEQ ID NO: 23 - protein, 97 aa, Homo sapiens
EVQLVQSGGGLVHPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 24 - protein, 96 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPP